(19) United States
(12) Patent Application Publication
(10) Pub. No.: US 2021/0136328 A1
(43) Pub. Date: May 6, 2021

(54) ENDOSCOPIC GRABBER WITH CAMERA AND DISPLAY

(71) Applicant: Saunders Midwest LLC, Chicago, IL (US)

(72) Inventors: Michael R. BARTHEL, Prospect Heights, IL (US); Douglas A. SPITLER, Naperville, IL (US); Dennis W. GILLOGLY, Sugar Grove, IL (US); Li FENG, Hangzhou City (CN)

(73) Assignee: Saunders Midwest LLC, Chicago, IL (US)

(21) Appl. No.: 17/087,802

(22) Filed: Nov. 3, 2020

Related U.S. Application Data
(60) Provisional application No. 62/930,311, filed on Nov. 4, 2019.

(51) Int. Cl. H04N 7/18; A61B 1/005; A61B 1/05; A61B 1/00; H04N 5/225; B25J 1/02; B25J 15/10; B25J 19/00

(52) U.S. Cl. CPC H04N 7/183; A61B 1/0051; A61B 1/05; A61B 1/00052; B25J 19/0029; A61B 1/00087; H04N 5/2252; B25J 1/02; B25J 15/106; A61B 1/00114

(57) ABSTRACT

An endoscopic grabber includes a proximal housing, a distal assembly, a flexible shaft extending between the proximal housing and the distal assembly, a flexible member within the flexible shaft having a distal end portion connected to the distal assembly and a proximal end portion connected to the proximal housing, and a camera within the distal assembly. A distal end portion of the flexible member is movably disposed within the distal assembly. A proximal end portion of the flexible member is connected to an actuator within the proximal housing. Actuation of the proximal end portion moves the distal end portion of the flexible member such that one or more elongate arms extending from the distal end portion deforms to form part of a grabber. The camera is configured to capture an image including a tip portion of the elongate arm. The elongate arms are configured to grab a target object.

### CROSS-REFERENCE TO RELATED APPLICATIONS

This application claims priority to U.S. Provisional Patent Application No. 62/930,311, entitled “ENDOSCOPIC GRABBER WITH CAMERA AND DISPLAY,” filed Nov. 4, 2019, which is related to PCT International Application No. PCT/US18/64519, entitled “ENDOSCOPIC GRABBER WITH WIRELESS CAMERA AND COMPACT EXTENSIBLE CAMERA,” filed Dec. 7, 2018, which claims priority to U.S. Provisional Application Ser. No. 62/595,668, entitled “ENDOSCOPIC GRABBER WITH WIRELESS CAMERA AND COMPACT EXTENSIBLE CAMERA,” filed Dec. 7, 2017, the disclosure of each of which is incorporated herein by reference in its entirety.

### BACKGROUND

The embodiments described herein relate to grabbing tools and extensible viewing devices. More specifically, embodiments described herein relate to grabbing tools having viewing capabilities including hand-operated grabbing tools combined with one or more endoscopic cameras, as well as to compact extensible viewing devices.

Known grabbing tools for allowing operators to reach, grab, and/or interact with an object via a hand-operated assistive devices are directed to performing motor functions alone, such as grabbing an object, without providing additional functionality. These devices rely on the user being able to view the target object sufficiently well with the naked eye to reach and manipulate the object, as well as the nearby environment sufficient well to navigate to the object. These tools include tools to grab items located beyond the user's reach, such as items located in tight or hard-to-reach locations. These tools also include extensible tools, such as wrenches, screw drivers and magnetic tips located at the end of an extension.

Many known tools for allowing operators to view a target area, such as endoscopes or borescopes, are similarly directed to performing viewing functions alone without providing additional functionality. These devices permit a user to guide a lens or camera to a hard-to-reach area and either take pictures of the target area or send video signals to viewer device seen by the user. Such known tools include camera mounts and endoscopic viewing devices. However, such known devices often provide only viewability of the target object or target area. As such, the user is required to perform subsequent actions to interact with an object viewed initially using the viewing device, such as to grab or manipulate a target object thereafter using a second tool without viewability assistance.

Further, many known viewing, display or inspection tools are overly complicated or expensive and are not easily combined with an assistive tool, such as with a grabber tool. Conventional viewing tools include expensive, specially designed electronic devices that provide particular types of viewing functions. For example, some known conventional medical endoscopes are designed to provide customized viewing for medical diagnoses or treatments. However, they are not well suited, and would be overly expensive to use, for both viewing a target object and manipulating the object with an extensible assist tool, such as to find a dropped bolt while working on a device. As another example, conventional industrial viewers are known that are designed for inspecting the integrity of a structure or for evaluating a necessary repair in construction industries. Likewise, these devices are not well suited, and would be overly expensive to use, for viewing a common or household target object to grab with a grabber tool.

Thus, a need exists for improved grabber tools, devices, and methods that provide viewing, lighting, or sensing features in combination with motility features of a grabber tool. Further, a need exists for simple and inexpensive devices for providing viewability in hard-to-reach locations for a user as needed without specialized viewing devices.

### SUMMARY

This summary introduces certain aspects of the embodiments described herein to provide a basic understanding. This summary is not an extensive overview of the inventive subject matter, and it is not intended to identify key or critical elements or to delineate the scope of the inventive subject matter.

In some embodiments, an apparatus includes a proximal control assembly and a distal assembly. The proximal control assembly includes a proximal housing and an actuator. The distal assembly includes an outer housing, an inner housing movably disposed within the outer housing, an electronic device disposed within a bore of the inner housing, and multiple elongate arms coupled to the inner housing. At least a portion of each of the elongate arms is disposed between an inner surface of the inner housing and the electronic device. A distal end portion of a flexible member is coupled to the inner housing, and a proximal end portion of the flexible member is coupled within the control assembly. An electrical wire has a distal end portion coupled to the electronic device and proximal end portion coupled within the control assembly. Actuation of the actuator moves the flexible member to cause the inner housing to move within the outer housing between a first position and a second position. A distal end portion of each elongate arm from the multiple elongate arms is in a first configuration within the outer housing when the inner housing is in the first position and is in a second configuration outside of the outer housing when the inner housing is in the second position. Simultaneously, a distal end of the electronic device is in a first location within the outer housing when the inner housing is in the first position, and the distal end of the electronic device is in a second location within the outer housing when the inner housing is in the second position, the second location is distal of the first location.

In some embodiments, an apparatus, such as an endoscopic grabber apparatus, includes a proximal housing including an actuator, a distal assembly, a flexible shaft, and a flexible member movably disposed within the flexible shaft. The distal assembly includes an outer housing, an inner housing, a set of elongate arms, and an electronic device. The inner housing is movably disposed within the outer housing, and the inner housing defines a bore. The set of elongate arms is coupled to an outer surface of the inner housing such that at least a portion of each elongate arm is within a volume between the outer surface of the inner housing and an inner surface of the outer housing. The electronic device is coupled within the bore of the inner housing. A proximal end portion of the flexible shaft is coupled to the proximal housing and a distal end portion of the flexible shaft is coupled to the outer housing. A distal end portion of the flexible member is coupled to the inner housing, and a proximal end portion is coupled to the actuator. The actuator is configured to move the flexible member to cause the inner housing to move within the outer housing between a first position and a second position. The distal end portion of each elongate arm from the plurality of elongate arms is in a first configuration within the outer housing when the inner housing is in the first position and is in a second configuration outside of the outer housing when the inner housing is in the second position. A distal tip of each elongate arm from the plurality of elongate arms extends outside of the outer housing when the inner housing is in the second position.

In some embodiments, the electronic device is a camera, a light emitting device, or an ultrasonic device. In other embodiments, the electronic device can be any sensing device, such as an infrared sensor, an optical sensor, a temperature sensor, pressure (e.g., sound pressure level) sensor, a biological sensor, a gas sensor, a radiation sensor, or the like. In some embodiments, the electronic device can include a wireless network interface configured to transmit a short-range wireless signal associated with an image or a signal received and/or produced by the electronic device.

In some embodiments, an endoscopic grabber apparatus includes a proximal housing, a distal housing, and a flexible shaft extending between the housings and connected to each housing. The endoscopic grabber apparatus further includes a flexible member within the flexible shaft having a distal end portion connected to the distal housing, and a proximal end portion connected to the proximal housing. The flexible member further includes a proximal end portion coupled to an actuator disposed within the proximal housing. Actuation of the proximal end portion of the flexible member moves a distal end portion of the flexible member within the distal housing to deform an elongate arm extending from the distal end portion. The apparatus includes a camera within the distal housing that can receive an image that includes an end portion of the elongate arm. In some embodiments, the camera includes an optical sensor within the distal housing coupled with an electronic module within the proximal housing. In some embodiments, the electronic module is coupled with a power source and a wireless interface configured to transmit the image to an electronic device.

In some embodiments, the actuation of the proximal end portion produces linear movement of the distal end portion. The linear movement of the distal end portion radially deforms a tip portion of the elongate arm to rotate away from a longitudinal direction of the elongate arm prior to the actuation. In some embodiments, the endoscopic grabber includes a plurality of elongate arms, and actuation of the distal end portion radially deforms tip portions of the plurality of elongate arms. The tip portions of the plurality of elongate arms can grab a target object in a central region disposed between the elongate arms. In some embodiments, the camera image can include the tip portions of the plurality of elongate arms. In some embodiments, a line segment can be determined that extends between each of the tip portions to a central point located between the tip portions, and the camera image can show the line segments in the camera image as virtual line segments. In some embodiments, the virtual line segments can be shown as virtual cross-hairs to help guide a user during grabbing or manipulation operations for the target object.

In some embodiments, a compact extensible camera device includes an extensible handle portion and a camera portion. The extensible handle portion can be arranged to move between a retracted, compact position and a plurality of extended positions. The camera portion can be attached and removed from the extensible handle portion and can include internal storage for storing images captured by the camera. In some embodiments, the extensible handle portion can be formed as a telescoping handle having a plurality of nested segments concentrically disposed within each other in a telescoping arrangement.

Other devices, systems, components, features, implementations, methods, and/or products according to embodiments will be or become apparent to one with skill in the art upon review of the following drawings and detailed description. It is intended that all such additional devices, systems, components, features, implementations, methods, and/or products be included within this description, be within the scope of this disclosure.

### BRIEF DESCRIPTION OF THE DRAWINGS

FIG. 1 is a perspective view of an endoscopic grabber device according to an embodiment.

FIG. 2 is a front perspective view of the embodiment of FIG. 1 showing the endoscopic grabber in an extended position.

FIG. 3 is a side view of the embodiment of FIG. 1 showing the endoscopic grabber in an extended position.

FIG. 4 is a side view of the extended position of the endoscopic grabber of FIG. 3 shown with the distal housing removed.

FIG. 5 is a side view of the embodiment of FIG. 1 showing the endoscopic grabber in a retracted position.

FIG. 6 is a side view of the retracted position of the endoscopic grabber of FIG. 5 shown with the distal housing removed.

FIG. 7 is a side view of the endoscopic grabber of FIG. 1 showing the endoscopic grabber in a retracted position.

FIG. 8 is lengthwise cross-sectional view of the endoscopic grabber of FIG. 7 showing the endoscopic grabber in a retracted position.

FIG. 9 is a lengthwise cross-sectional view of the endoscopic grabber of FIGS. 1-7 showing the endoscopic grabber in an extended position.

FIG. 10A is an enlarged view of the distal end portion of the endoscopic grabber shown in FIG. 9.

FIG. 10B is a zoomed view of region K indicated in FIG. 10A of a portion of the distal end portion of the endoscopic grabber shown in FIG. 9.

FIG. 10C is cross-sectional view of a portion of the distal end portion of the endoscopic grabber shown in FIG. 9 as viewed from line X-X shown in FIG. 10B.

FIG. 10D is an enlarged view of the proximal end portion of the endoscopic grabber shown in FIG. 9.

FIG. 11 is a front view of a viewer device that can be used with the endoscopic grabber device shown in FIGS. 1-10D according to an embodiment, which shows an example camera view from the endoscopic grabber.

FIG. 12A is a side view of an endoscopic grabber according to an embodiment in a retracted position, which is shown with the flexible shaft following an example curvilinear path.

FIG. 12B is a side view of a portion of the endoscopic grabber of FIG. 12A in a retracted position, in which the flexible shaft follows an example linear path.

FIG. 12C is a lengthwise cross-sectional view of the proximal end portion of the endoscopic grabber of FIG. 12A.

FIG. 13A is a side view of an endoscopic grabber according to another embodiment.

FIG. 13B is a perspective end view of the endoscopic grabber of FIG. 13A.

FIG. 14 is a cross-sectional side view of the endoscopic grabber of FIG. 13A shown in a first configuration.

FIG. 15 is a cross-sectional side view of a portion of the endoscopic grabber of FIG. 13A shown in the first configuration.

FIG. 16 is an enlarged cross-sectional view of a proximal end portion of the endoscopic grabber of FIG. 13A shown in the first configuration.

FIG. 17 is an enlarged cross-sectional view of a distal end portion of the endoscopic grabber of FIG. 13A shown in the first configuration.

FIG. 18 is a cross-sectional side view of the endoscopic grabber of FIG. 13A shown in a second configuration.

FIG. 19 is a cross-sectional side view of a portion of the endoscopic grabber of FIG. 13A shown in the second configuration.

FIG. 20 is an enlarged cross-sectional view of a proximal end portion of the endoscopic grabber of FIG. 13A shown in the second configuration.

FIG. 21 is an enlarged cross-sectional view of a distal end portion of the endoscopic grabber of FIG. 13A shown in the second configuration.

FIG. 22 is an enlarged view of a portion of the distal end portion of the endoscopic grabber of FIG. 13A shown in the second configuration and with an outer housing removed.

FIG. 23 is a side view of a portion of the proximal end portion of the endoscopic grabber of FIG. 13A with the housing open showing interior components of the control portion of the device.

FIGS. 24 and 25 are each a different partially disassembled view of the endoscopic grabber of FIG. 13A illustrating an interior portion of the housing of the proximal control assembly.

FIG. 26 is a perspective view of a distal end portion of the endoscopic grabber of FIG. 13A with an alternative outer housing and inner housing included within the distal assembly with a portion of the outer housing removed.

FIG. 27 is an enlarged view of a portion of the endoscopic grabber with alternative outer and inner housings shown in FIG. 26.

FIG. 28 is a perspective view of a distal end portion of the endoscopic grabber of FIG. 26 illustrating partial actuation of the elongate arms extending partially out of the outer housing.

FIG. 29 is perspective view of a distal end portion of the endoscopic grabber of FIG. 26 illustrating actuation of the elongate arms extending out of the outer housing.

FIG. 30 is a cross-sectional view illustrating the outer housing and the inner housing disposed therein.

FIG. 31 is a perspective view of a portion of the distal assembly of the endoscopic grabber of FIG. 13A.

FIG. 32 is an enlarged partially exploded perspective view of a portion of the distal assembly of the endoscopic grabber of FIG. 13A.

FIG. 33 is a perspective view of a portion of an electrical wire and a portion of a flexible member shown partially within an outer wrap, of the endoscopic grabber of FIG. 13A.

FIGS. 34 and 35 each illustrate a portion of a proximal control assembly of the endoscopic grabber of FIG. 13A, with FIG. 35 showing the mounting member removed from the guide housing for illustration purposes.

FIG. 36A is a perspective view of an endoscopic grabber device, according to another embodiment.

FIG. 36B is a side view of a portion of the endoscopic grabber device of FIG. 36A.

FIG. 37 is a perspective view of the control assembly of the endoscopic grabber device of FIG. 36.

FIG. 38 is a front view of the control assembly of the endoscopic grabber of FIG. 36.

FIG. 39 is a rear view of the control assembly of the endoscopic grabber of FIG. 36.

FIG. 40 is a right side view of the control assembly of the endoscopic grabber of FIG. 36.

FIG. 41 is a left side view of the control assembly of the endoscopic grabber of FIG. 36.

FIG. 42 is a top view of the control assembly of the endoscopic grabber of FIG. 36.

FIG. 43 is a bottom view of the control assembly of the endoscopic grabber of FIG. 36.

### DETAILED DESCRIPTION

The embodiments described herein can advantageously be used in a variety of endoscopic grabber devices and compact extensible camera devices, tools and components, and associated methods and operations. In particular, the devices described herein can be integrated endoscopic grabber and extensible viewer devices, accessories and components for viewing target objects in difficult to reach locations, as well as for grabbing or manipulating target objects while concurrently viewing the objects and/or the corresponding environments.

Various example features, aspects, configurations, components, assemblies, and arrangements are generally described herein pertaining to an endoscopic grabber device, such as example endoscopic devices 100, 200 and 300, which can be used to grab and/or manipulate a target object while also viewing, lighting, and/or sensing the object and corresponding environment. Embodiments of endoscopic grabber devices described herein are each configured to operate as an integrated endoscopic grabber device that an operator can use to reach, grab, and optionally manipulate a target object while concurrently viewing the object and nearby environment. The user can simply maneuver a distal grabber portion of the endoscopic grabber device into a position close to the target object with the aid of concurrent views from a camera disposed on the distal grabber portion. Further, the user can simply actuate the proximal handle portion of the endoscopic grabber device when positioned with respect to the target object to operate the distal grabber portion to grab and/or manipulate the target object. The grabbing and/or manipulation operations regarding the target object can be greatly enhanced by providing the user with concurrent views from the camera.

As used herein, the term “about” when used in connection with a referenced numeric indication means the referenced numeric indication plus or minus up to 10 percent of that referenced numeric indication. For example, the language “about 50” covers the range of 45 to 55. Similarly, the language “about 5” covers the range of 4.5 to 5.5.

The term “flexible” in association with a part, such as a mechanical structure, component, or component assembly, should be broadly construed. In essence, the term means the part can be repeatedly bent and restored to an original shape without permanently deforming the part. Certain flexible components can also be resilient. For example, a component (e.g., a flexure) is said to be resilient if possesses the ability to absorb energy when it is deformed elastically, and then release the stored energy upon unloading (i.e., returning to its original state). Many “rigid” objects have a slight inherent resilient “bendiness” due to material properties, although such objects are not considered “flexible” as the term is used herein.

As used in this specification and the appended claims, the word “distal” refers to direction towards a target object, and the word “proximal” refers to a direction away from the target object. Thus, for example, the end of an endoscopic grabber device that is closest to the target object or target surface would be the distal end of the endoscopic grabber device, and the end opposite the distal end (i.e., the handle end manipulated by the user) would be the proximal end of the endoscopic grabber device.

Further, specific words chosen to describe one or more embodiments and optional elements or features are not intended to limit the invention. For example, spatially relative terms such as “beneath”, “below”, “lower”, “above”, “upper”, “proximal”, “distal”, and the like may be used to describe the relationship of one element or feature to another element or feature as illustrated in the figures. These spatially relative terms are intended to encompass different positions (i.e., translational placements) and orientations (i.e., rotational placements) of a device in use or operation in addition to the position and orientation shown in the figures. For example, if a device in the figures is turned over, elements described as “below” or “beneath” other elements or features would then be “above” or “over” the other elements or features. Thus, the term “below” can encompass both positions and orientations of above and below. A device may be otherwise oriented (e.g., rotated 90 degrees or at other orientations) and the spatially relative descriptors used herein interpreted accordingly. Likewise, descriptions of movement along (translation) and around (rotation) various axes includes various spatial device positions and orientations.

Similarly, geometric terms, such as “parallel”, “perpendicular”, “round”, or “square”, are not intended to require absolute mathematical precision, unless the context indicates otherwise. Instead, such geometric terms allow for variations due to manufacturing or equivalent functions. For example, if an element is described as “round” or “generally round,” a component that is not precisely circular (e.g., one that is slightly oblong or is a many-sided polygon) is still encompassed by this description.

In addition, the singular forms “a”, “an”, and “the” are intended to include the plural forms as well, unless the context indicates otherwise. The terms “comprises”, “includes”, “has”, and the like specify the presence of stated features, steps, operations, elements, components, etc. but do not preclude the presence or addition of one or more other features, steps, operations, elements, components, or groups.

As used herein, the term “camera” in the context of an electronic device refers to an electronic optical device for a capturing an image, which can include one or more sensor components for receiving the image and/or one or more components for interpreting, transforming, managing, storing or otherwise processing the image to be in a viewable format. As such, a camera can include one or more components separated from each other, such as an electronic image sensor at a first location that captures an image, and an electronic control module or other processing component for processing the captured electronic image into a viewable format, which can be at a second location that is collocated with or spaced apart from the first location.

Unless indicated otherwise, the terms apparatus, device, tool, etcher and variants thereof, can be interchangeably used.

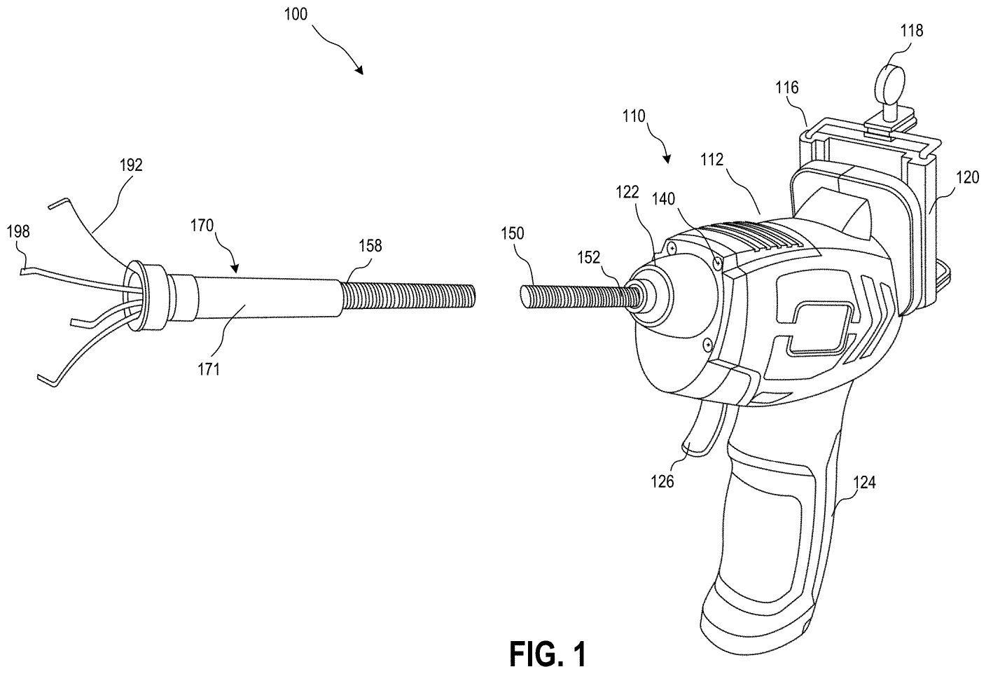

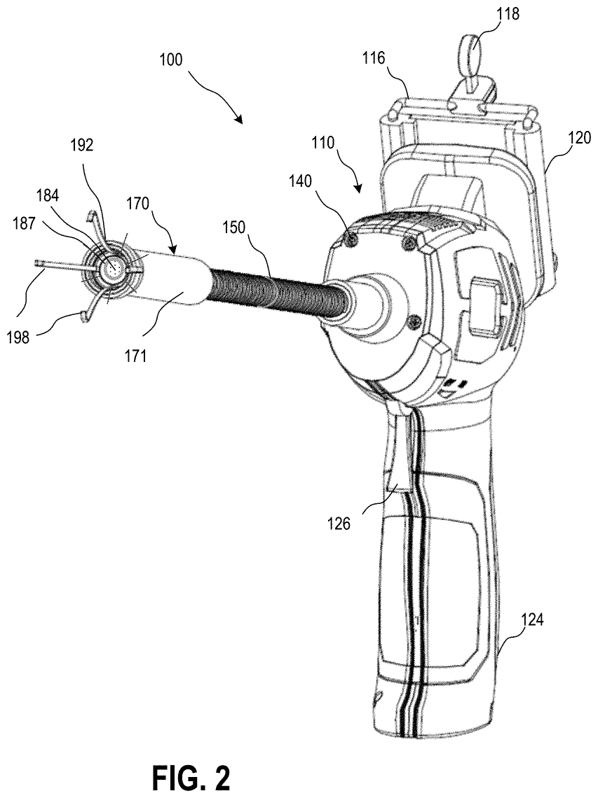

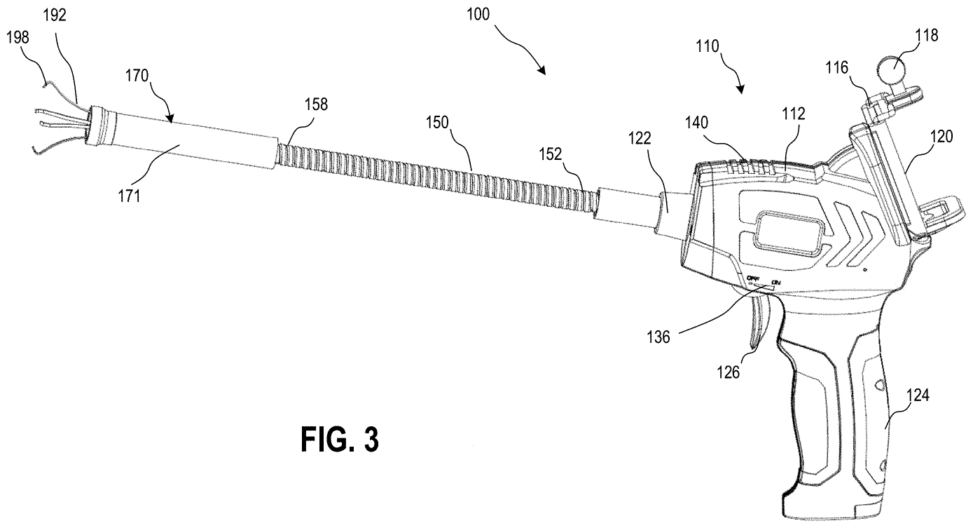

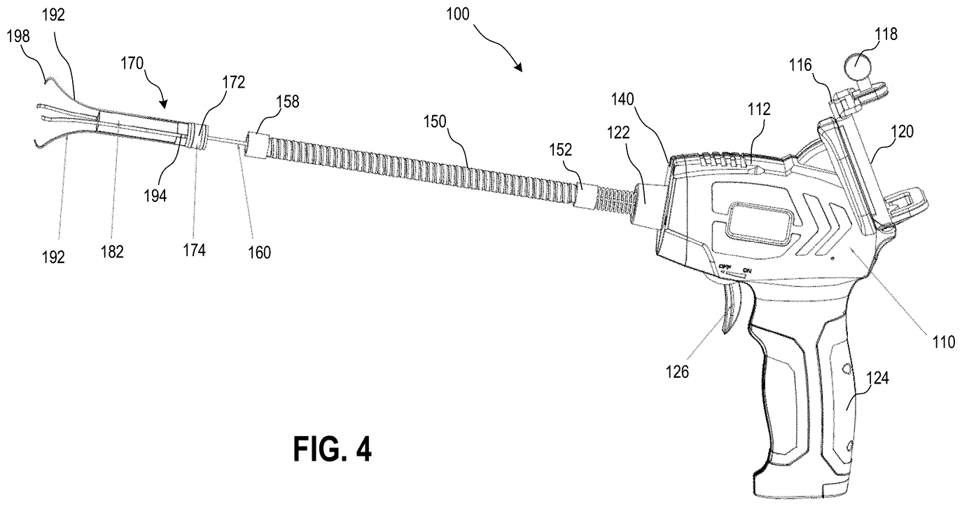

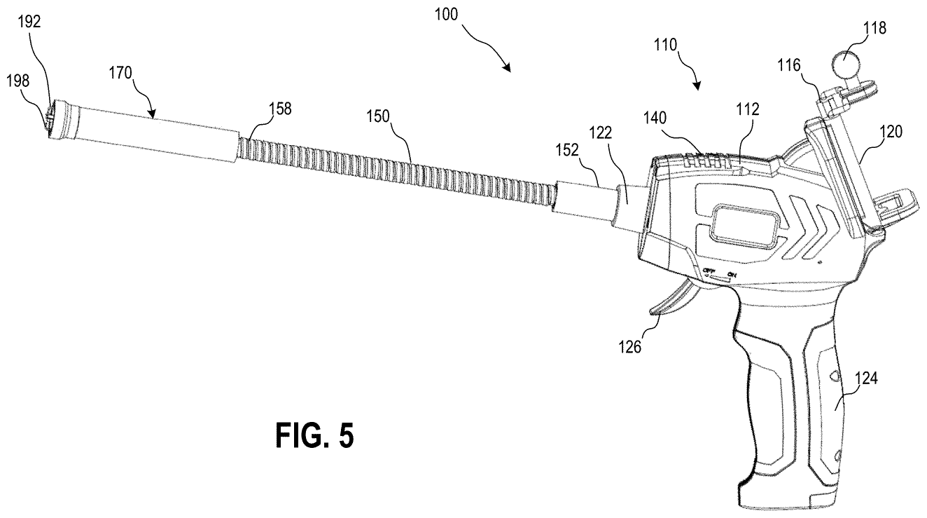

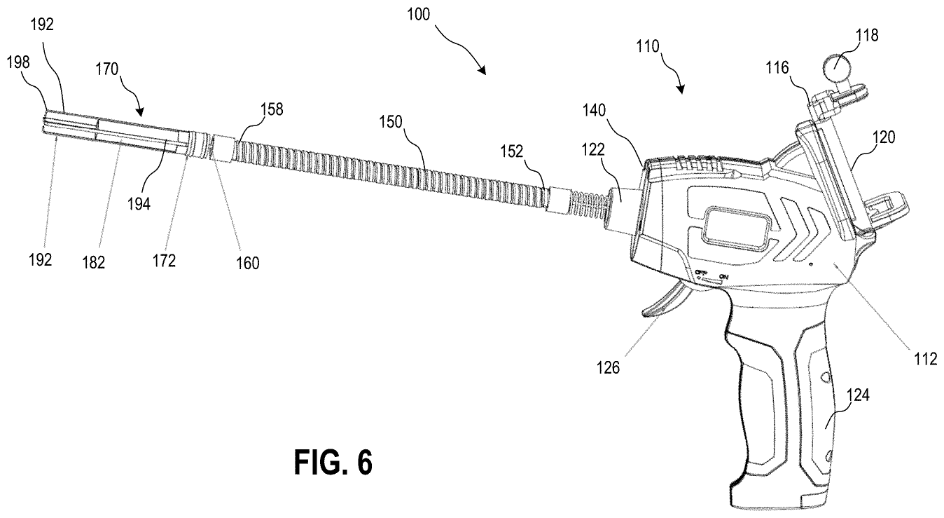

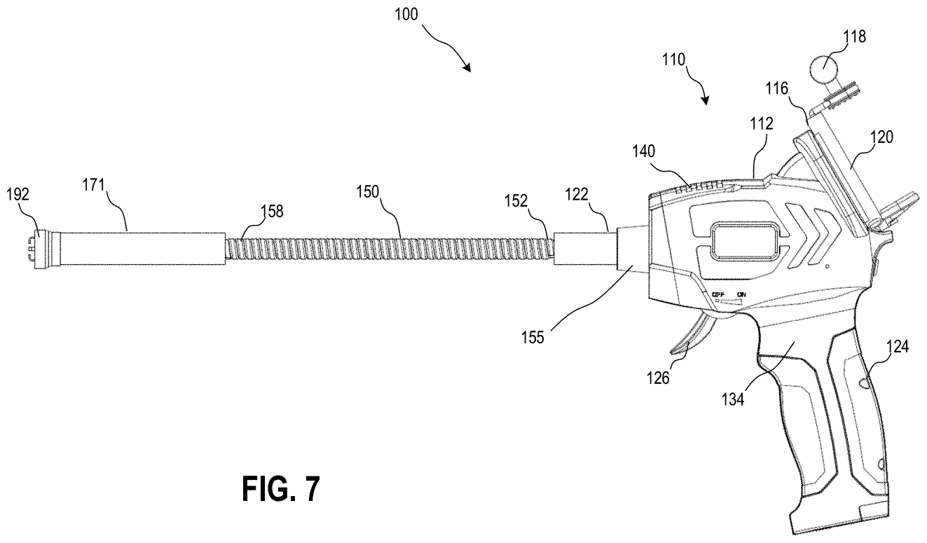

FIGS. 1-10D show an example endoscopic grabber device 100 according to an embodiment. The endoscopic grabber device 100 includes a control assembly 110 having a proximal housing 112 and the control components therein, a distal assembly 170, a flexible shaft 150 extending between the control assembly 110 and the distal assembly 170, and a flexible member 160 located within the flexible shaft 150. The flexible shaft 150 has a distal end portion 158 connected to the distal assembly 170, and a proximal end portion 152 connected to the proximal housing 112. The control assembly 110 includes an actuator 126 coupled to the proximal housing 112 that is configured to actuate movement of the flexible member 160 as described herein. The flexible member 160 is movably disposed within the flexible shaft 150. A distal end portion 168 of the flexible member 160 is coupled to the distal assembly 170, and a proximal end portion 162 of the flexible member is coupled (via a mounting member 156) to the actuator 126 of the proximal housing 112. The actuator 126 is configured to move the flexible member 160 to actuate movement of the distal assembly 170.

The flexible member 160 can be formed as a flexible wire. The flexible shaft 150 can be formed from a flexible metal shaft, a flexible elastomeric shaft, or the like, and defines an internal channel for the flexible member 160 to translate therein while permitting the shaft to bend and flex as needed to reach a target object during use. The flexible member 160 further includes or is coupled to the mounting member 156 or push wire mounting member 156 and a spring 154 is disposed at a proximal end portion of the flexible member 160. The spring 154 biases the flexible member 160 in the direction of the proximal housing 112. As such, actuation of the actuator 126 acts to move the mounting member 156 against the bias of the spring 154, and the spring urges the mounting member 156 and flexible member 160 toward the distal housing 170 when released. Thus, actuation of actuator/trigger 126 advances the flexible member forward into an extended arrangement, and releasing the actuator/trigger 126 biases the flexible member to rearward into an extracted arrangement.

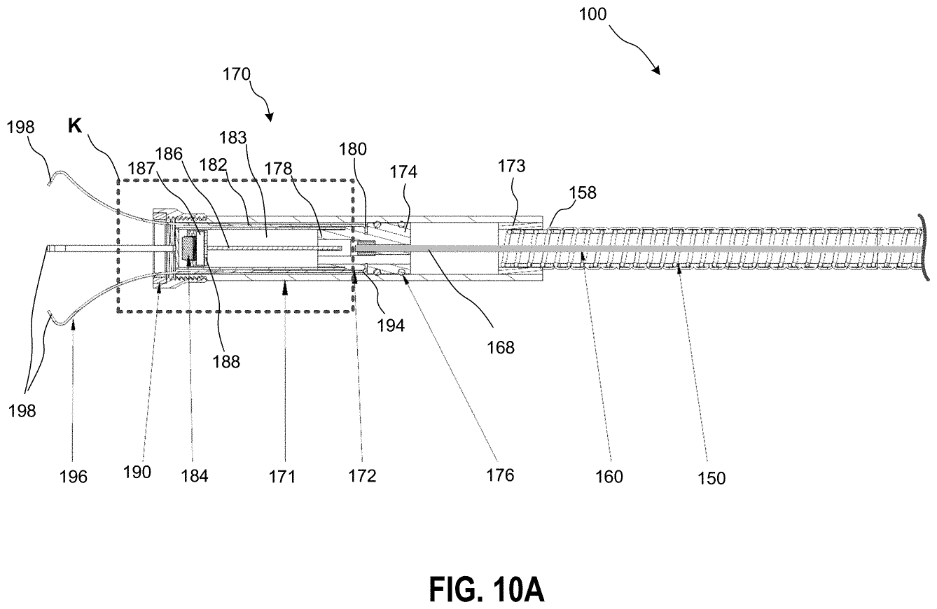

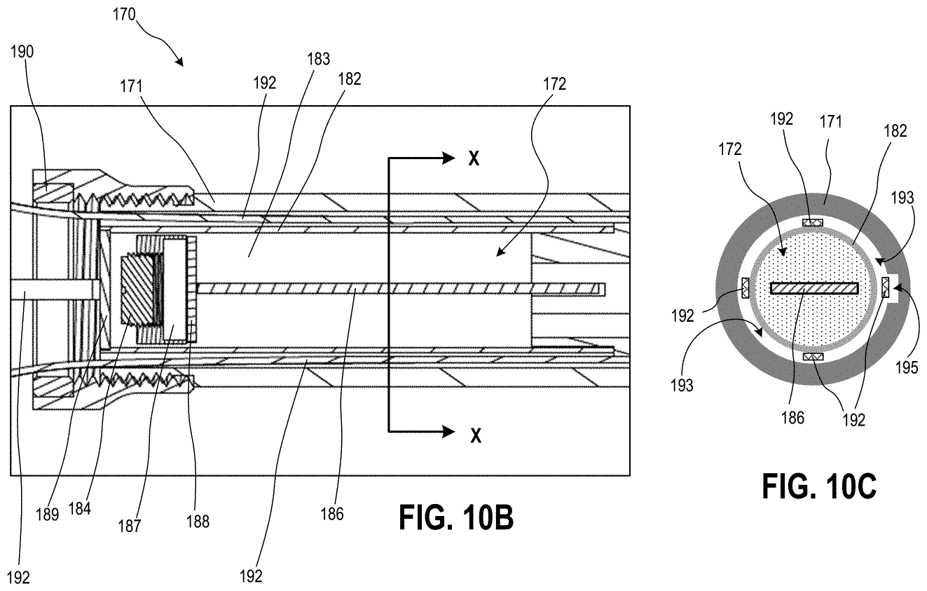

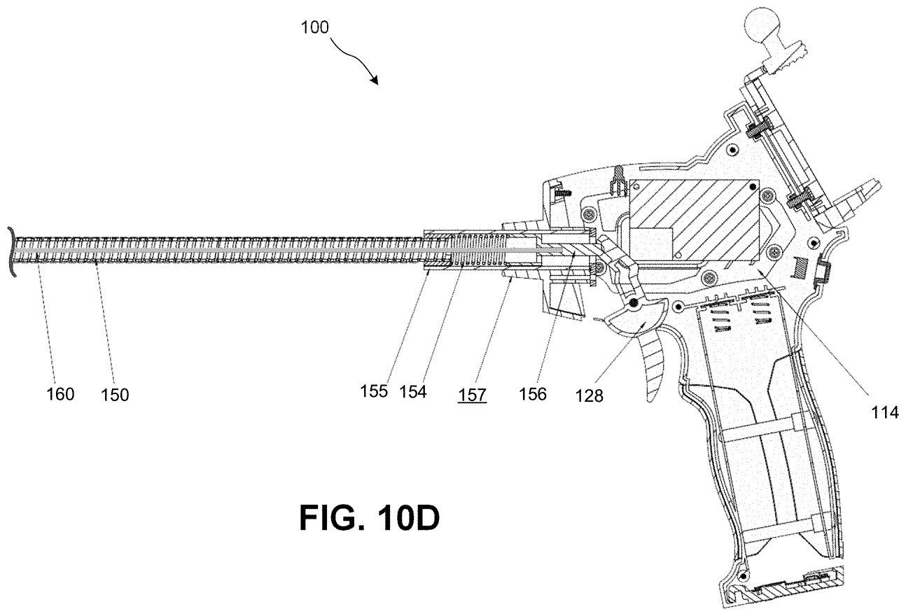

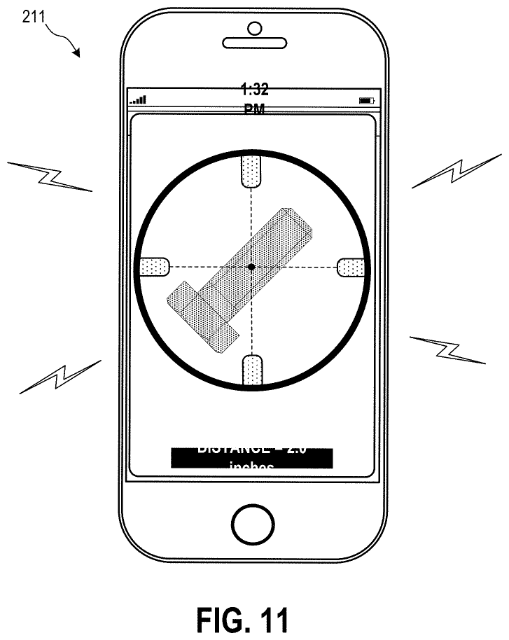

Referring to FIGS. 10A-10C, the distal assembly 170 includes an outer housing 171, an inner housing 182 movably disposed within the outer housing, a plurality of elongate arms 192 coupled to an outer surface of the inner housing 182, and an electronic device 184. A coupling portion 173 at a proximal end portion of the outer housing 171 connects the distal end portion 158 of the flexible shaft to the outer housing. In this manner, the outer housing 171 remains fixedly coupled to the flexible shaft 150 during operation of the device 100. Similarly stated, when the flexible member 160 is actuated, the outer housing 171 does not move relative to the flexible shaft 150.

The outer housing 171 defines an interior volume within which the inner housing 182 is located. As shown in FIG. 10C, the outer housing 171 can be a tubular housing such that the interior (or inner surface) of the outer housing is defined by an internal diameter of the outer housing 171. An exterior portion (or outer surface) of the inner housing 182 (and/or the movable member 172) is smaller than the interior of the outer housing such that the inner housing 182 fits within the outer housing 171. As such, the inner housing 182 is configured to move or translate within the outer housing 171 and, thereby, operate as a portion of a movable carrier for the distal assembly 170, as described below. More specifically, referring to FIG. 10C, an annular gap 193 within which the elongate arms 192 are maintained is defined between the exterior of the inner housing 182 and the interior of the outer housing 171. In some embodiments the inner surface of the outer housing 171 can define one or more guide channels 195. Such guide channels can have an elongate orientation (i.e., can extend along a longitudinal axis of the distal assembly 170) to guide each of the elongate arms 192 during actuation and movement between the first and second configurations. For example, FIG. 10C shows a single guide channel 195 oriented in an elongate orientation with one of the elongate arms 192 as an example to illustrate the optional use of guide channels. In other embodiments, the outer housing 171 does not define any guide channels, which arrangement allows the inner housing 182 and/or the elongate arms 192 to rotate relative to the outer housing 171 about the longitudinal axis of the distal assembly 170. In other embodiments, the outer housing 171 can include multiple guide channels (e.g., one for each of the elongate arms 192).

The distal end portion of the outer housing 171 includes a magnetic attachment member (also referred to as the magnet) 190 that can assist with attracting or coupling to a target object. Although the magnet 190 is shown as being threadedly coupled to the distal end portion of the outer housing 171, in other embodiments, a magnet can be coupled to the outer housing 171 by any suitable means, such as by a press fit, an adhesive, or the like. In yet other embodiments, the outer housing 171 need not include the magnetic attachment member 190.

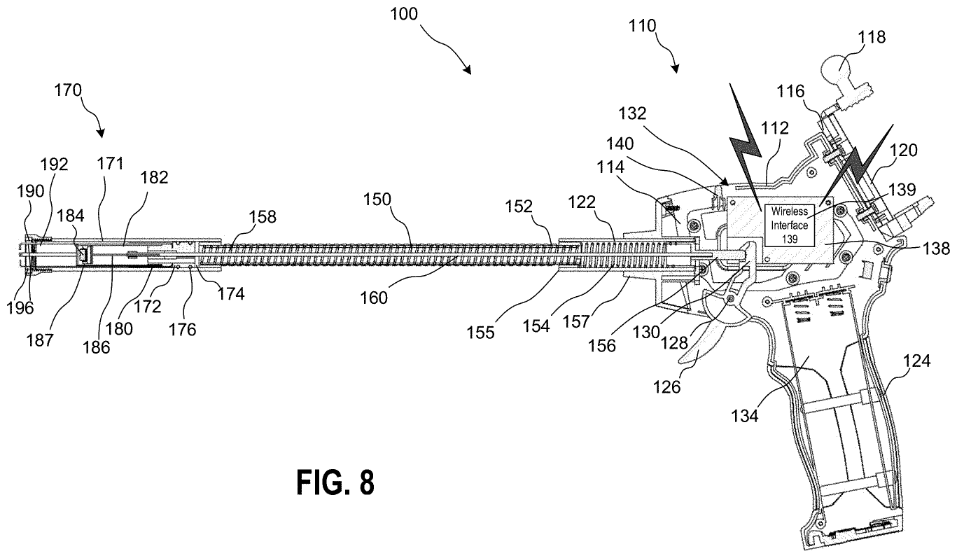

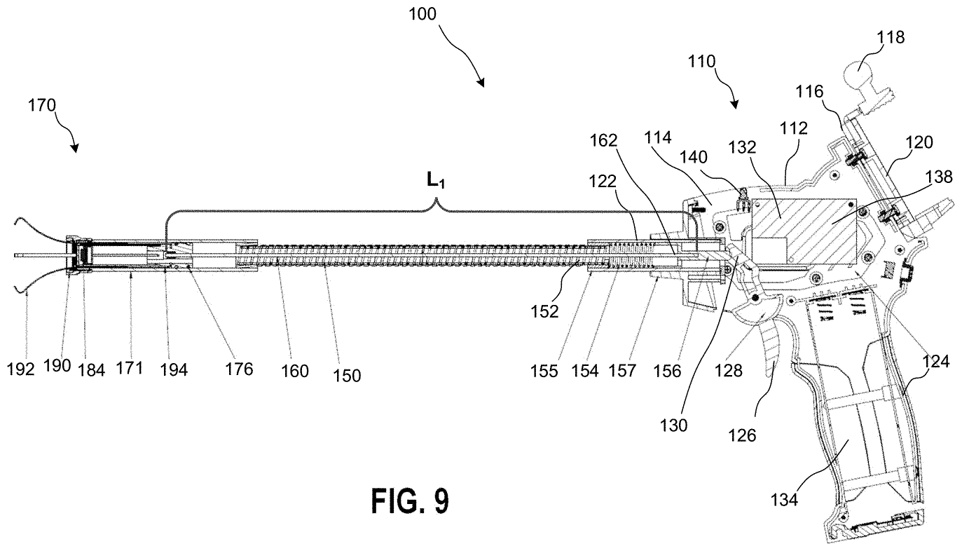

As further shown in FIG. 10C, the interior of the inner housing 182 defines a bore 183, within which the electronic device 184 is coupled. The proximal end portion of the inner housing 182 includes or is coupled to a movable member 172 (which functions as a junction member between the flexible member 160, the electronic device 184, and the elongate arms 192). As shown in FIG. 10A, a distal end portion 178 of the movable member 172 is aligned with and coupled to a proximal end portion of the inner housing 182 within the interior of the outer housing 171. As such, the movable member 172 and the inner housing 182 operate together to form a translatable movable carrier (e.g., to carry the electronic device 184 and/or the elongate arms 192) within the outer housing 171. Although the inner housing 182 and the movable member 172 (or junction member) are shown as being separate components that are joined together, in other embodiments, the inner housing 182 and the movable member 172 can be monolithically constructed. A proximal end portion 174 of the movable member 172 is coupled to a distal end of the flexible member 160, which extends from the distal end 158 of the flexible shaft and into the interior of the outer housing 171, to couple to the proximal end portion of the movable member. As such, the flexible member 160 extends between the movable member 172 and the actuator 126 through the flexible shaft 150, such that movement of the actuator 126 causes the inner housing 182 and the movable member 172 to move together within the outer housing 171 between a first position (FIG. 8) and a second position (FIGS. 9 and 10A).

As noted above, the movable member 172 joins the inner housing 182 to the flexible member 160, the electronic device 184, and the elongate arms 192. Specifically, the proximal end portion 174 of the movable member 172 includes an elongate arm connection 180 through which the elongate arms 192 are coupled to the movable member 172. Although the elongate arm connection 180 shows a portion of each elongate arm 192 being embedded within the movable member 172, in other embodiments, the elongate arms 192 can be coupled to the movable member 172 and/or the inner housing 182 by any suitable mechanism (e.g., by a weld joint, an adhesive joint, or the like). In some embodiments, the elongate arms 192 can be monolithically constructed with the movable member 172. The proximal end portion 174 of the movable member is also attached to the distal end 168 of the flexible member 160 and includes sealing rings 176. The sealing rings 176 are disposed around the movable member 172 within the bore 182 of the distal housing retain the movable member 172 in the sliding arrangement within the annular volume 193 defined within the outer housing 171. The sealing rings 176 can be formed from polymeric materials that provide a low-friction connection within the distal housing to enable sliding movement therein and that also prevent dust, dirt or other foreign materials from entering the interior of the outer housing 171.

As shown in FIG. 10B, the electronic device 184 is attached to the distal end portion 178 of the movable member 172 and is oriented to produce light, capture images, and/or sense conditions when the device is actuated. The electronic device 184 includes a mounting member 188 and electrical connections 186 that are coupled to the movable member 172. The mounting member 188 can include any suitable mechanism for securing the electronic device 184 within the bore of the inner housing 182. For example, in some embodiments, the mounting member 188 can include shock-absorbing properties, an interference fit portion, or any other suitable features to retain the electronic device 184 at the desired position within the inner housing 182. The electrical connections 186 can include one or more wires that electrically connect the electronic device 184 to the electronic controller 138. The wires can be collocated with the flexible member 160 and can extend through the flexible shaft 150 along with the flexible member. The wires (along with the electrical connections 186) can allow power to be conveyed from the battery 134 to the electronic device 184. The wires (along with the electrical connections 186) can also allow control signals and/or data signals to be transferred between the electronic device 184 and the electronic controller 138.

The electronic device 184 can be any suitable device that can produce light and/or sense conditions adjacent the distal end of the device 100. For example, in some embodiments, the electronic device can be a camera, a light emitting device, or an ultrasonic device. In other embodiments, the electronic device can be any sensing device, such as an infrared sensor, a temperature sensor, a radiation sensor, a gas sensor, or an optical sensor. In some embodiments, the electronic device 184 can include (or be coupled to) a wireless network interface configured to transmit a short-range wireless signal associated with an image or a signal received and/or produced by the electronic device 184.

As shown in FIG. 10A, each of the elongate arms 192 includes a proximal end 194 and a distal end 196. Each of the distal ends includes a distal tip 198. Referring to FIGS. 8 and 10A-10C, as described above, the proximal end 194 of each of the plurality of elongate arms 192 is coupled to an outer portion of the movable member 172 at the coupling portion 180 of the movable member. Each of the elongate arms extends from the respective proximal end 194 to the respective distal end 196 of the elongate arm. As can be seen in FIG. 8, the distal end 196 of each elongate arm 192 from the plurality of elongate arms is configured to be at first position within the outer housing 171 when the inner housing is in a first, non-deployed position. When the elongate arms 192 are in the first position, the distal tip 198 of each elongate arm extends from the distal-most surface of the outer housing 171 by a first distance. In other embodiments, however, the distal tip 198 of each elongate arm can be fully retracted within the outer housing 171 when the elongate arms are in the first position. Moreover, when the elongate arms 192 are in the first position, they are also in a first (deformed) configuration. Specifically, the each of the elongate arms is within the annular volume 193 and is therefore deformed by the inner housing 182 and the outer housing 171 to be in a substantially linear configuration. Similarly stated, when in the retracted position, each of the elongate arms 192 extends in a longitudinal direction that is generally parallel with the longitudinal axis of the distal assembly 170.

As shown in FIG. 10A, when the device 100 is actuated, the distal tip 198 of each elongate arm 192 from the plurality of elongate arms is at a second position extending outside of the outer housing 171. More specifically, because the elongate arms 192 are coupled to the inner housing 182 (via the movable member 172), when the movable member 172 and inner housing 182 are moved within the outer housing 171, the elongate arms 192 relative to the outer housing 171 to deploy the elongate arm 192. When the elongate arms 192 are in the second (deployed) position, the distal tip 198 of each elongate arm extends from the distal-most surface of the outer housing 171 by a second distance, greater than the first distance. The elongate arms 192 are each made from a flexible material (e.g., spring steel) that is arranged to rotate or flex outward away from each other and away from their longitudinal direction as they are translated forward out of the bore during actuation of the device 100. The distal ends 196 are arranged to form a set of inward directed hook-like shapes at each tip 198. As such, when the actuator 126 is actuated, the tip 198 of each elongate arm 192 is directed inward toward a central region disposed the elongate arms and a central point between the tips. In this manner, when the device 100 is returned to its undeployed state, the tips 198 can grasp an object within the central region.

In addition to moving the elongate arms 192 between their first position and their second position, actuation of the device 100 also move the electronic device 184 between its first position within the outer housing 171 to its second position within the outer housing 171. Specifically, because the electronic device 184 is fixedly coupled within the inner housing 182, movement of the inner housing 182 and the movable member 172, which causes movement of the elongate arms 192, also causes the electronic device 184 to be moved outward from its first (inward) position to its second (outward) position.

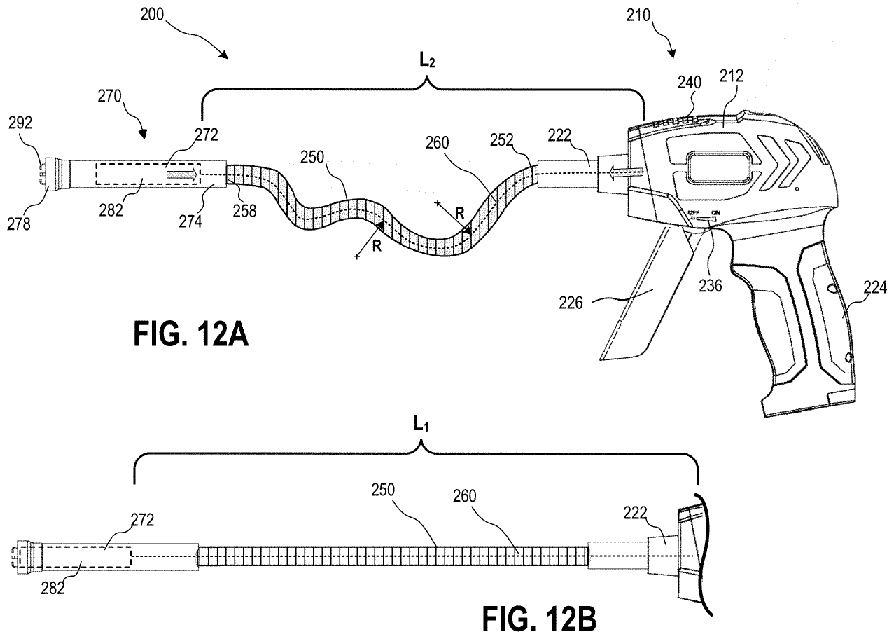

The relative position of the electronic device 184 and the tips 198 when the elongate arms are deployed can cooperatively function to provide advantageous data collection. For example, as described herein, in embodiments in which the electronic device 184 is a camera, the camera can receive an image that includes an end portion 196 of the elongate arms. In addition, the electronic control 138 can be configured to identify the tips 198 in the image. The electronic control 138 can further be configured to identify the central point between the tips and a line segment between each tip and the central point, and to show the line segments and/or central point as virtual features in the display device as discussed further below along with FIG. 11.

As best seen in FIGS. 8, 9 and 10D, the control assembly 110 includes the proximal housing 112, a rear handle or grip portion 124, a shaft connection 155, a manipulator portion 122 that receives the proximal portion of the flexible member 160, an actuator or trigger 126, and an electronics module 132. The proximal housing 112 defines an internal volume 114 therein, in which the electronics module 132 is secured. The proximal housing 112 further includes a viewer mounting portion 116 that is arranged to removably retain a viewing device or phone device, such as viewing device 211 shown in FIG. 11. The mounting portion 116 includes a flat face 120 for receiving the viewing device 211, and a mounting knob 118 for removably retaining the viewing device during use.

The rear handle or grip portion 124 is configured as a handle that can be easily held by a user and allow the user to manipulate the endoscopic grabber device 100 during use. The actuator or trigger 126 is disposed on an upper, front region of the grip portion 124 and located for easy access by a user's index finger. The actuator or trigger 126 includes a pivot portion 128 that is rotatably mounted within the internal volume 114 of the proximal housing. A lever end 130 of the actuator or trigger 126 is located on an internal end of the pivot portion 128 adjacent to a mounting end 156 at the proximal end portion 162 of the flexible member 160. The lever end 130 is arranged in a cantilever arrangement with the exposed trigger end of the actuator 126 on the pivot portion 128. Actuation of the trigger end of the actuator 126 rotates the pivot portion 128 to move the lever end 130 to rotate away from the proximal housing toward the distal housing 170 and push the flexible member 160 to translate forward within the flexible shaft 150 in the direction of the distal housing 170. A face of the lever end 130 can be curved to maintain good contact with the mounting end 156 of the flexible member 160 during actuation.

The shaft connection 155 is disposed on a forward portion of the proximal housing 112 to securely connect the proximal end portion 152 of the flexible shaft 150 to the proximal housing. The manipulator portion 122 is disposed within the shaft connection 155 to receive the proximal end portion 152 of the flexible shaft 150 and to retain components of the proximal end portion of the flexible shaft, which are discussed in more detail below along with the flexible shaft.

The electronics module 132 includes a power source (or battery, not shown), a control switch 136, the electronic controller 138 (which can include a processor), and one or more lights 140. As shown in FIGS. 8, 9 and 10D, the power source can include a battery within a battery storage region 134 defined within the proximal housing. In other configurations, the power source can include components for coupling to an alternating current power supply (not shown) in addition to components for a battery power source or as an alternative to a battery power source. Such configurations can include a power cord and transformer, as well as a charger for charging a battery. The control switch 136 can include a simple on/off switch, as well as optional settings for activating the lights 140 and/or the electronic device 184. The controller 134 can include a processor, a memory, and a wireless network interface.

The processor can be configured to run and/or execute application modules, processes and/or functions associated with the device 100. For example, the processor can be configured to run and/or execute an image capture module that facilitates capturing and processing of an image produced by the electronic device 184. The processor can be, for example, a Field Programmable Gate Array (FPGA), an Application Specific Integrated Circuit (ASIC), a Digital Signal Processor (DSP), and/or the like. The processor can be configured to retrieve data from and/or write data to a memory device (not shown). As described herein, in some embodiments, the processor can cooperatively function with the network interface device and/or a radio to provide signals to communicatively couple the electronics module 132 to a remote computing device (e.g., such as the device 211 via wireless communication) and/or any other computing entity via a network. In some embodiments, the processor is a Bluetooth® low energy (BLE) processor, such as The Texas Instruments® CC2540 series of processors, the Broadcom® BCM43341 processor, and/or any other processor suitable or configured specifically to execute the Bluetooth® v4.0 low energy stack.

The memory (not shown) can be, for example, random access memory (RAM), memory buffers, hard drives, databases, erasable programmable read only memory (EPROMs), electrically erasable programmable read only memory (EEPROMs), read only memory (ROM), flash memory, hard disks, floppy disks, cloud storage, and/or so forth. In some embodiments, the memory stores instructions to cause the processor to execute modules, processes and/or functions associated the device 100. For example, the memory can store instructions to cause the processor to execute the image capture module.

Referring to FIG. 8, the electronic controller 138 includes a wireless interface or radio 139, which can be any suitable communication device and can be a part of the overall processor architecture of the electronic control 138, (e.g., a part of a Bluetooth® processor). In other embodiments, the radio or wireless interface 139 can be distinct from a processor of the electronic control. In some embodiments, a short-range radio link can be established between the electronic module 132 and a mobile electronic device, such as a mobile device 211 discussed below along with FIG. 11. For example, the electronic module 132 and/or the electronic controller 138 and the mobile device 211 can be paired via the Bluetooth® wireless protocol. Similarly stated, the electronic module 132 and/or the electronic control 138 and the mobile device 211 can be paired via a wireless protocol that facilitates the transmission of signals within a range of approximately 700 meters or less (i.e., a Class 3 radio) and/or having a frequency within the range of 2400 MHz and 2480 MHz. In such an embodiment, as described in further detail herein along with FIG. 11, the electronic module 132 and/or the electronic controller 138 can be operable to send and/or receive data from the mobile device 211 related to an image acquire by the device, such as from the electronic device 184.

Referring to FIG. 9, the proximal end portion 162 of the flexible member 160 is connected to the actuator 126 disposed within the proximal housing 112. Actuation of the proximal end portion of the flexible member moves the distal end portion of the flexible member 160 within the distal assembly 170, which permits deformation of each of the plurality of elongate arms 192 extending from the distal end portion to flex outward into a gripping position.

In operation, actuation of the actuator 126 moves the mounting member 156 in the proximal housing 112 as discussed above to push the flexible member 160 forward within the flexible shaft 150 toward the distal housing 170. Movement of the flexible member 160 correspondingly moves the movable member 172 forward within the distal housing 170, which advances the elongate arms 192 and the electronic device 184 forward within the distal housing 170. As the elongate arms 192 advance and extend outside of the outer housing 171, the elongate arms flex or rotate outward to increase the size of a central region disposed between the tips 198 of the elongate arm. In some embodiments, the advancement of the electronic device 184 also allows the central region produced between the tips 198 to be sensed by the electronic device 184 (e.g., viewed by the camera, in some embodiments). The endoscopic grabber device 110 can be advanced toward a target object (not shown) based on the camera view to place the target object within the central region. The user can release the actuator 126 to bias the flexible member 160 rearward and thereby collapse the tips 198 around the target object to grab the object. If the target object is magnetic, the optional magnet 190 can be used to grab the object via a magnetic connection alone or along with use of the elongate arms 192.

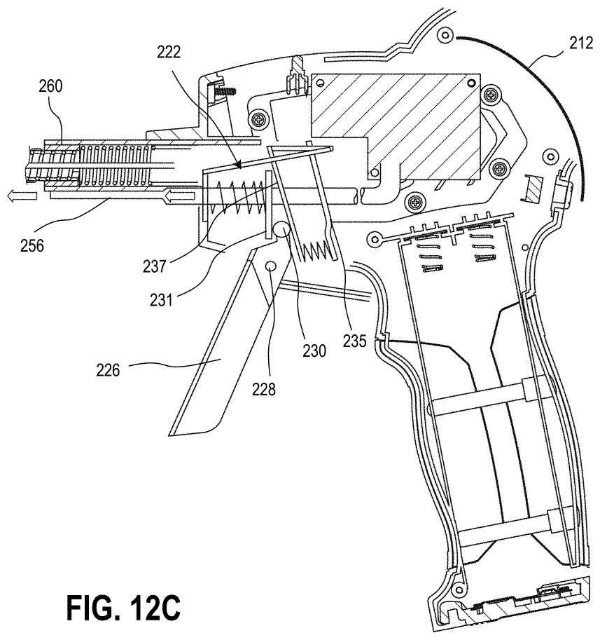

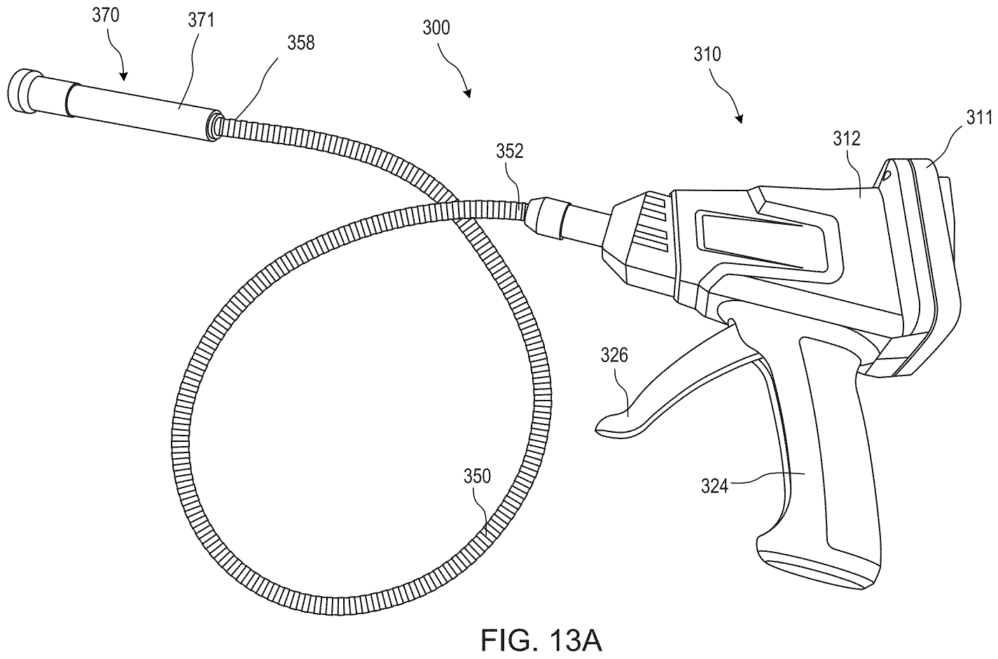

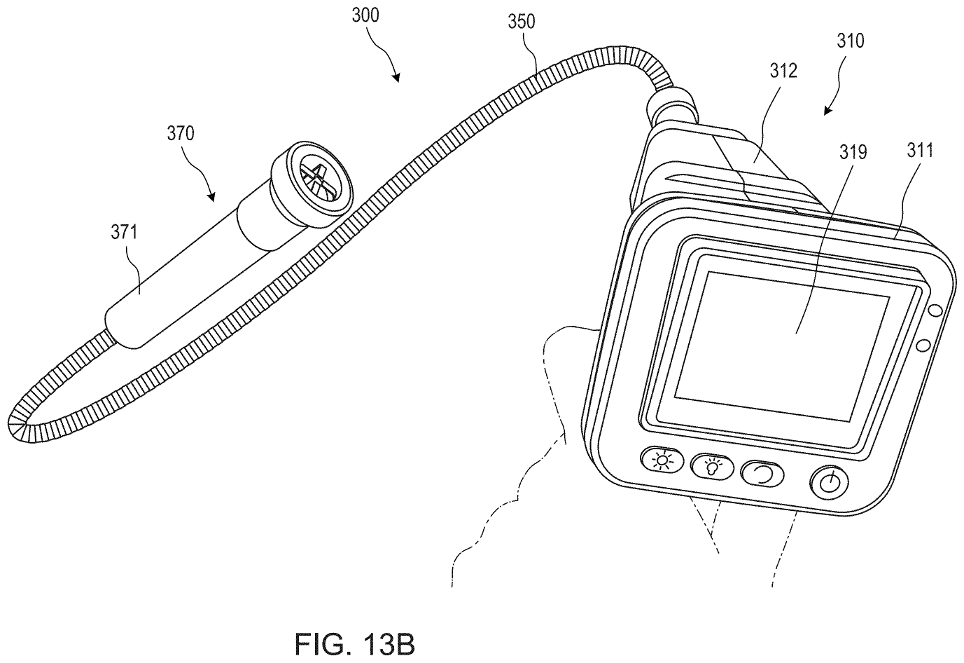

Referring now to FIG. 11, a mobile device is shown that can be used as a viewing device 211, including a display screen, in conjunction with an endoscopic grabber device, such as the example endoscopic grabber device 100 discussed above and/or example endoscopic grabber device 200 discussed below along with FIGS. 12A-12C. The viewing device 211 is shown as a portable phone device or mobile device (e.g., an iPhone®, an Android® device, a Windows® phone, a Blackberry® phone, etc.), but it is understood that various types of viewing devices can be used with endoscopic grabber devices discussed herein. Such viewing devices can include, for example, a tablet computer (e.g., an Apple iPad®, a Samsung Nexus® device, a Microsoft Surface® device, etc.), or a computer (e.g., a laptop, desktop, smart TV, etc.), and/or any other suitable computing entity. In some embodiments, the viewing device 211 includes a mobile phone device that has viewer application configured to connect with the electronic controller 138 of endoscopic grabber device 100 and display information received from the electronic device 184 on the display screen of the viewing device 211. The viewer application can be configured simply to display a view provided from the electronic controller 138 and/or the viewer application can be configured to modify the view as discussed further below, such as to add virtual line segments to provide a cross-hairs type view, show a virtual central point and/or to estimate distances to the tips 198 or the target object.

As shown in FIG. 11, in some embodiments, the viewing device 211 displays an image that is captured by the electronic device 184. The image can show the tips 198 of the elongate arms 192 along with the target object and corresponding environmental features within its view while the endoscopic grabber device 100 is activated and being used. The electronic controller 138 can be configured to identify the tips 198 in the captured image. Alternatively, the viewer application can be configured to identify the tips 198 in the captured image, and the tips 198 could optionally be highlighted or marked to aid the identification. The viewer application or the electronic controller 138 can further be configured to identify the central point between the tips and a line segment between each tip and the central point, and to show the line segments and/or central point as virtual features on the display device 211. These virtual features could provide a cross-hairs type view to aid the user with aligning the endoscopic grabber device 100 effectively for grabbing the target object.

In addition, the electronic controller 138 and/or the viewer application can be configured to estimate distances to the target object and/or the distance that the elongate arms 192 extend from distal end of the endoscopic grabber device 100. The size of the tips 198 can be known to the electronic controller 138 and/or the viewer application, which can be used to determine the distance that the tips are extended. Further, the electronic controller 138 and/or viewer application can compare the size of the target object being viewed with the size of the tips, as well as monitor the changing size of the object when approaching the target object, from which distances can be estimated. In addition, the distance that the elongate arms are extended can be monitored based on movement of the movable member, for example, which can provide additional information for estimating distances and/or the size of the target object.

Referring now to FIGS. 12A to 12C along with FIG. 9, an embodiment of an endoscopic grabber 200 is shown in FIGS. 12A to 12C. Endoscopic grabber 200 generally includes the same aspects and features as endoscopic grabber 100 discussed above, except as discussed herein. Referring to FIG. 12B the endoscopic grabber 200 includes a flexible member 260 located within a flexible shaft 250 that extends between a manipulator portion 222 of the proximal housing 212 and the movable member 272 of the distal assembly 270. When the flexible shaft 250 is maintained in a general straight configuration between the proximal housing and the distal assembly, as shown in FIG. 12B, a length, L₁, of the flexible member 260 is generally the same as that of the flexible shaft 250 between the endpoints of the flexible shaft (see also, the length L₁ shown in FIG. 9). As shown in FIG. 12A, however, the flexible shaft 250 is configured to have many different curvilinear arrangements as appropriate for following a path to gain access to a target object to grab, which changes the overall length of the flexible member 260 compared with the flexible shaft 250. Stated differently, bends and curves along the length of the flexible shaft 250 can induce tensile and compression forces, F, along the longitudinal axis of the flexible member 260, which can, under certain circumstances, cause the movable member 272 to bind within the distal assembly 270. Under such high bend circumstances, the actuation of an endoscopic grabber can become difficult due to the elongation of the flexible member 260.

Accordingly, the device 200 includes an actuator arrangement that allows for consistent actuation when the flexible shaft 250 is both straight and curved (in any amount). Referring to FIG. 12C, actuator 226 is configured to be adjustable with respect to flexible member 260 such that the flexible member 260 slidably engages an axial drive member 237 coupled to the longitudinal axis of the flexible member 226. When the actuator 226 rotates about the pivot 228, a drive lever 230 moves push ring 231 to translate along the longitudinal axis of axial drive member 237 and, thereby, move flexible member 260 toward the distal assembly 270. Axial drive member 237 can adjustably translate with respect to push ring or push plate 231 as compressive and tensile forces are encountered along the flexible member 260 responsive to curvilinear movements of the flexible shaft and flexible member. A user can further fine tune adjustment of the axial drive member 237 with respect to push ring 231 via angled release 235.

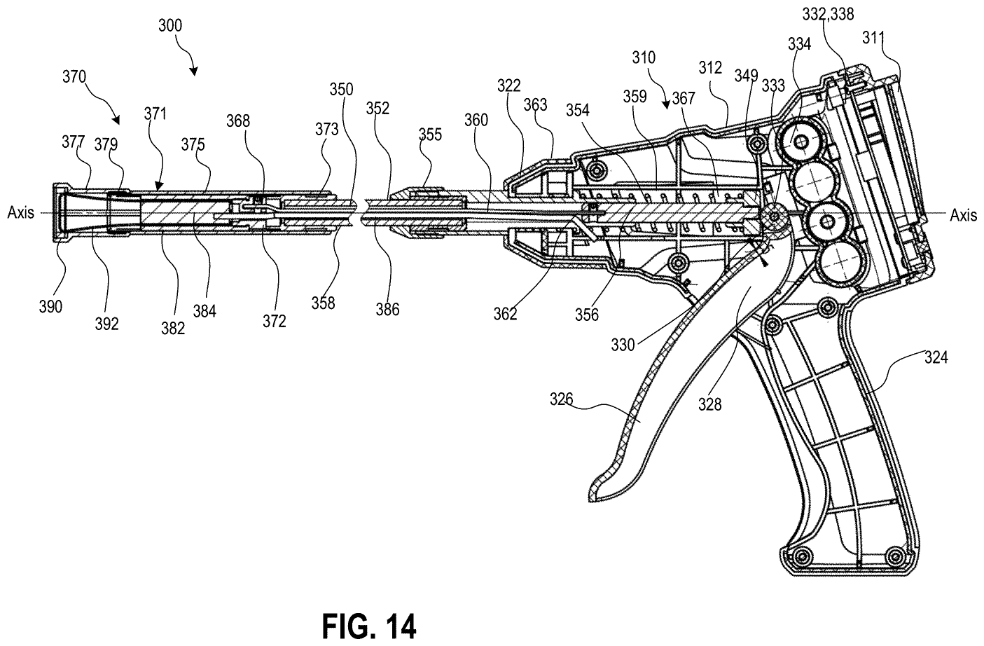

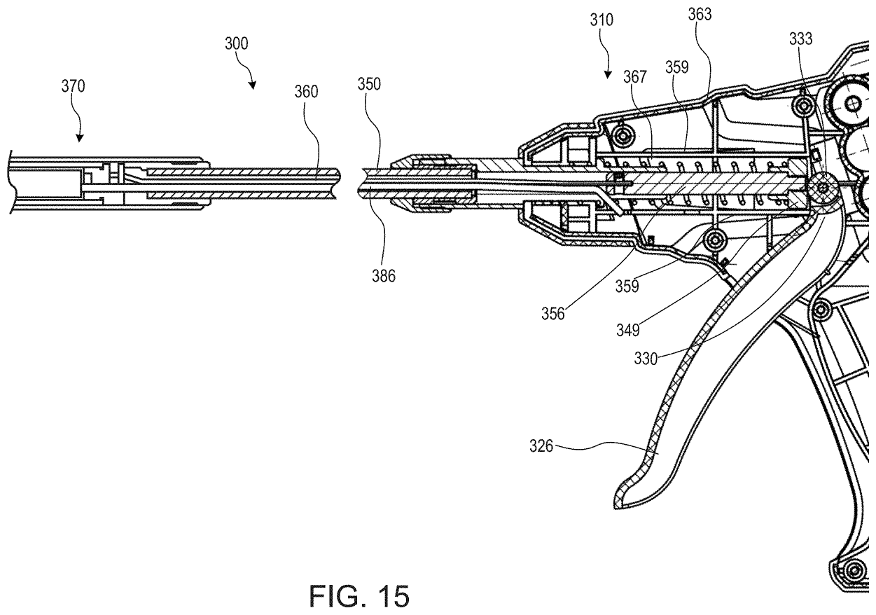

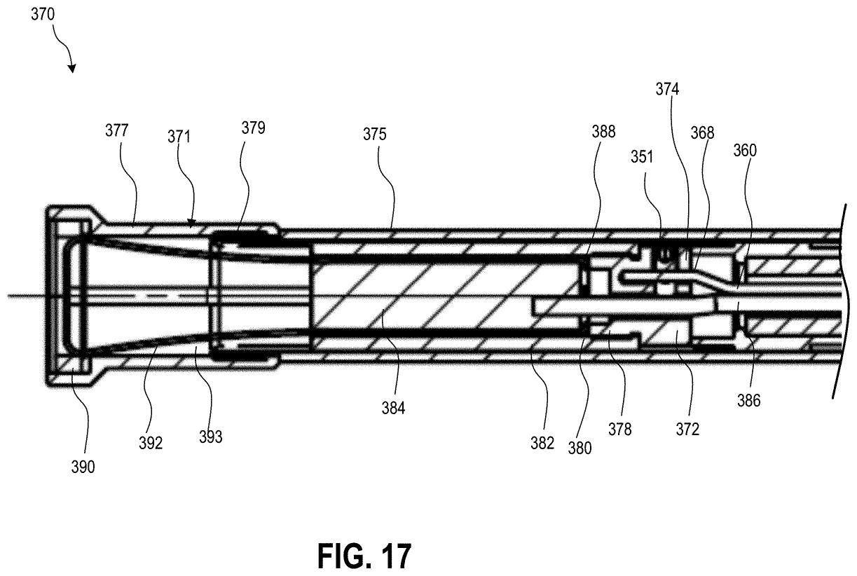

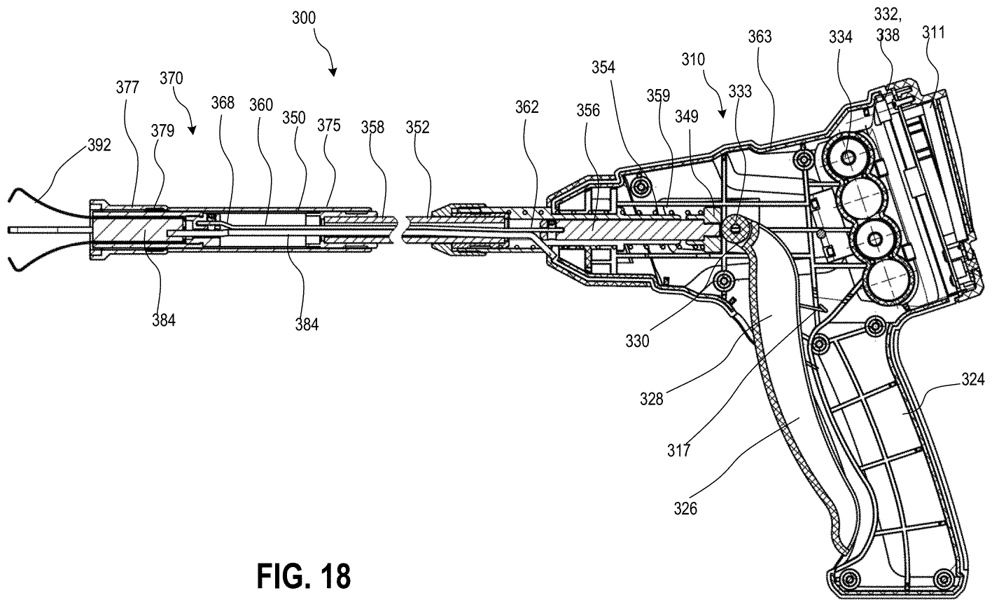

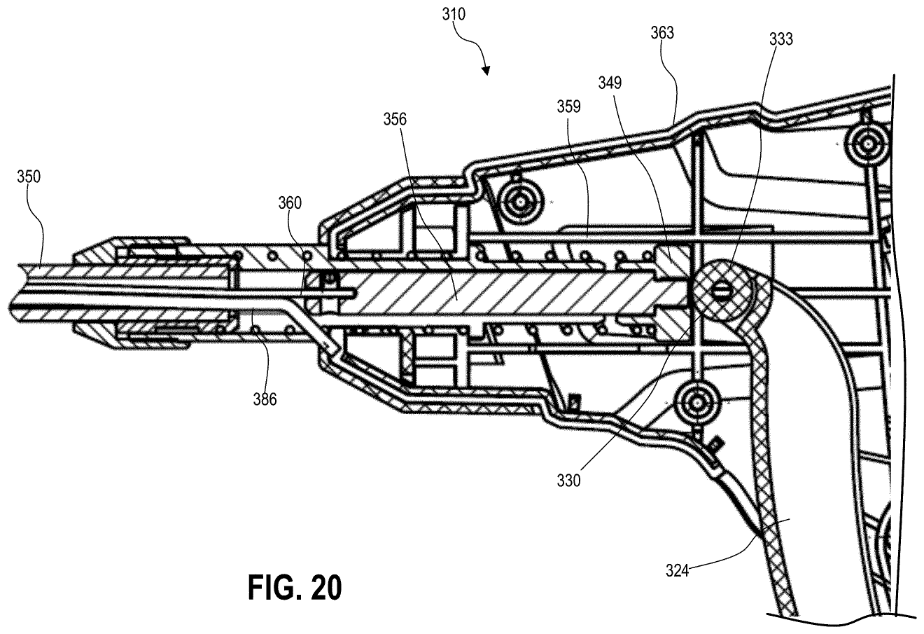

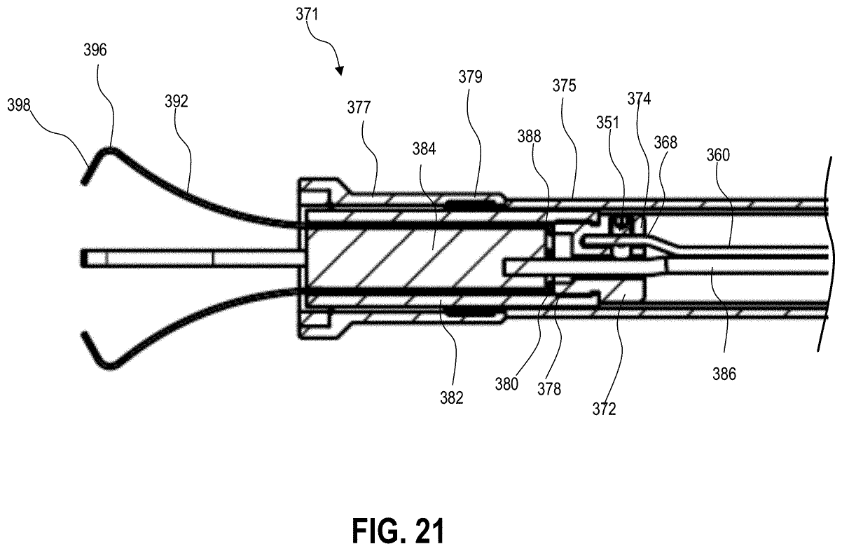

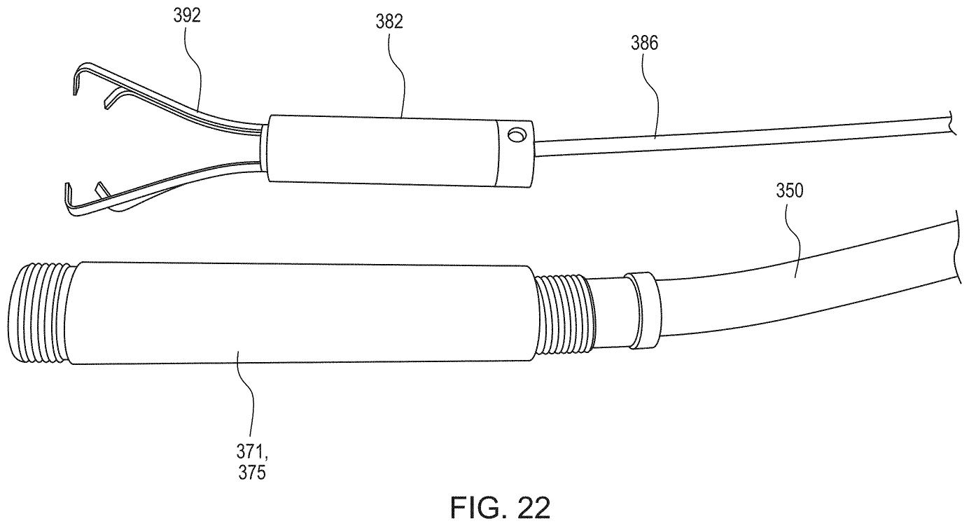

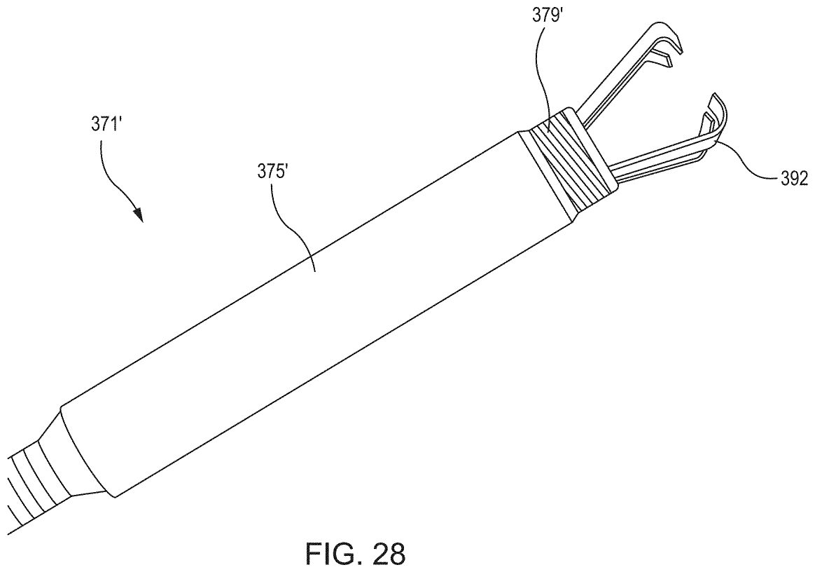

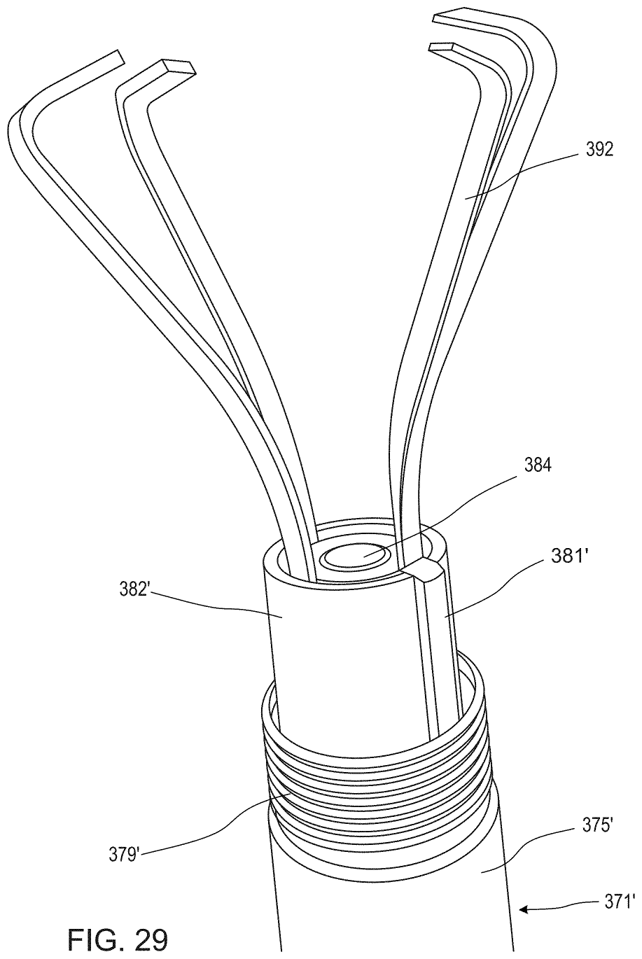

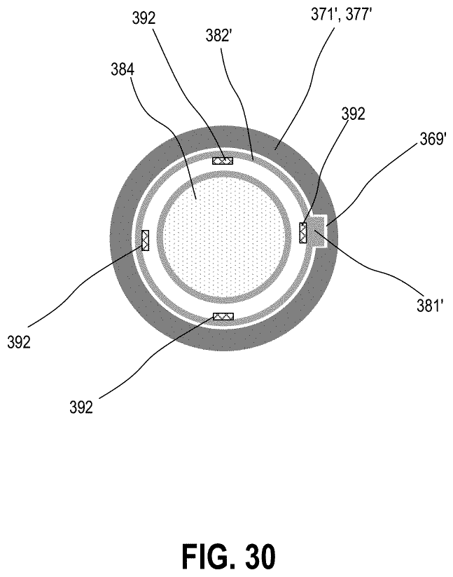

FIGS. 13A-35 illustrate another embodiment of an endoscopic grabber. An endoscopic grabber 300 can include the same or similar features and functions as described above for endoscopic grabber 100, and therefore some features and functions are not described in detail with reference to this embodiment. FIGS. 13A-17 illustrate components of the endoscopic grabber 300 in a retracted position and FIGS. 18-21 illustrate components of the endoscopic grabber 300 in an extended position.

The endoscopic grabber device 300 includes a control assembly 310 having a proximal housing 312 and the control components therein, a distal assembly 370, a flexible shaft 350 extending between the control assembly 310 and the distal assembly 370, and a flexible member 360 located within the flexible shaft 350. The flexible shaft 350 has a distal end portion 358 connected to the distal assembly 370, and a proximal end portion 352 connected to the proximal housing 312. In this embodiment, the control assembly 310 includes a display device 311 fixedly or permanently coupled to the proximal housing 312 and described in more detail below.

The control assembly 310 also includes an actuator 326 coupled to the proximal housing 312 via pivot pins 353 located at a pivot portion 328 and that are received within a pin shafts 313 of the housing 312 such that the actuator 326 can pivot relative to the housing 312, and a torsion spring 317 (see e.g., FIGS. 18, 24 and 25) is coupled between the actuator 326 and the housing 312 as described in more detail below. The actuator 326 is configured to actuate movement of the flexible member 360 as described herein. The flexible member 360 is movably disposed within the flexible shaft 350. A distal end portion 368 of the flexible member 360 is coupled to the distal assembly 370, and a proximal end portion 362 of the flexible member 360 is coupled to a mounting member 356 within the proximal housing 312. The actuator 326 is configured to move the flexible member 360 via the mounting member 356 to actuate movement of the distal assembly 370 as described in more detail below.

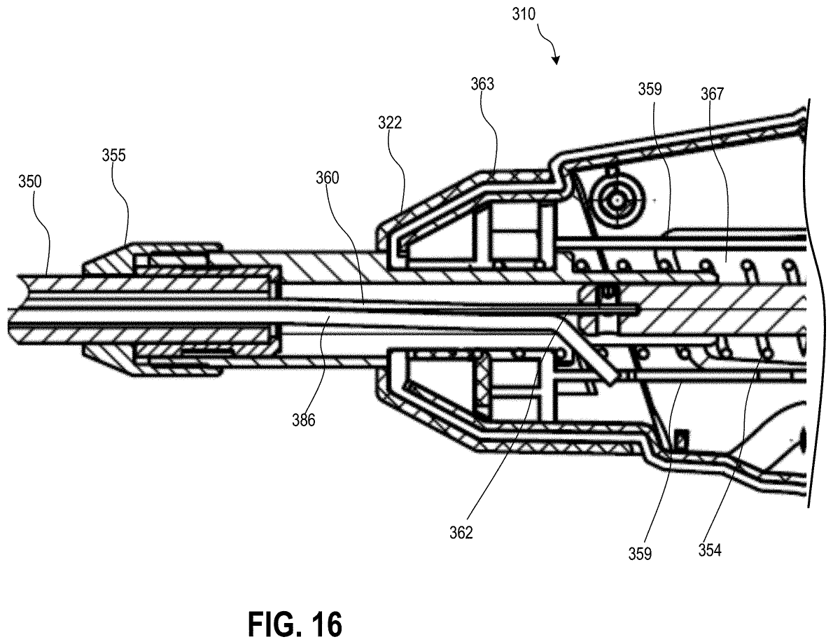

In some embodiments, the flexible member 360 can be formed as a flexible wire. The flexible shaft 350 can be formed from a flexible metal shaft, a flexible elastomeric shaft, or the like, and defines an internal channel for the flexible member 360 to translate therein while permitting the shaft to bend and flex as needed to reach a target object during use. As described above, the mounting member 356 is disposed at the proximal end portion 362 of the flexible member 360 and movably disposed within the flexible shaft 350. The mounting member 356 includes a guide portion 349 at a proximal end that engages the actuator 326 and travels within a guide passage 367 of the proximal housing 312, as described in more detail below. A spring 354 is disposed about and coupled to the mounting member 356 within the proximal housing 312. The spring 354 biases the flexible member 360 in a direction of the proximal housing 312 as shown in FIGS. 14-16. Upon actuation of the actuator 326, the mounting member 356 moves against the bias of the spring 354, and the mounting member 356 and flexible member 360 are urged in a direction toward the distal housing 370, as shown in FIGS. 18-20. Thus, actuation of actuator 326 advances the flexible member 360 into the extended position, as shown in FIGS. 18-20, and releasing the actuator 326 biases the flexible member into the retracted position, as shown in FIGS. 14-16.

The distal assembly 370 includes an outer housing 371 (see, e.g., FIGS. 13A and 13B), an inner housing 382 movably disposed within the outer housing, multiple elongate arms 392 coupled to the inner housing 382, and an electronic device 384. More specifically, as shown, for example, in FIGS. 14, 17, 18 and 21, the elongate arms 392 are disposed between an outer surface of the inner housing 382 and an outer surface of the electronic device 384. In addition, a coupling portion 373 at a proximal end portion of the outer housing 371 connects the distal end portion 358 of the flexible shaft 350 to the outer housing 371. In this manner, the outer housing 371 remains fixedly coupled to the flexible shaft 350 during operation of the device 300. Similarly stated, when the flexible member 360 is actuated, the outer housing 371 does not move relative to the flexible shaft 350.

The outer housing 371 defines an interior volume within which the inner housing 382 is located and can translate proximally and distally within the outer housing 371. In some embodiments, a lubricant is disposed between an outer surface of the inner housing 382 and an inner surface of the outer housing 371 to reduce or eliminate friction between the outer housing 371 and the inner housing 382 during movement distally and proximally. The outer housing 371 includes a first housing portion 375 threadably coupled to a second housing portion 377 at a threaded coupling joint 379. The outer housing 371 can be, for example, a tubular housing such that the interior (or inner surface) of the outer housing 371 is defined by an internal diameter of the outer housing 371. For example, the first housing portion 375 and the second housing portion 377 can each have an interior surface defined by an internal diameter of the first housing portion 375 and the second housing portion 377. In some embodiments, the internal diameter of the first housing portion 375 is the same as the internal diameter of the second housing portion 377. An exterior portion (or outer surface) of the inner housing 382 is smaller than the interior of the outer housing 371 (e.g., the interior of the first housing portion 375 and the interior of the second housing portion 377) such that the inner housing 382 fits within the outer housing 371. As such, the inner housing 382 is configured to move or translate within the outer housing 371 and, thereby, operate as a portion of a movable carrier for the distal assembly 370, as described below. In addition, with the elongate arms 392 coupled to the inner housing 382 and the electronic device 384 coupled to the inner housing 382, the elongate arms 392 and electronic device 384 both move or translate with the inner housing 382 relative to the outer housing 371.

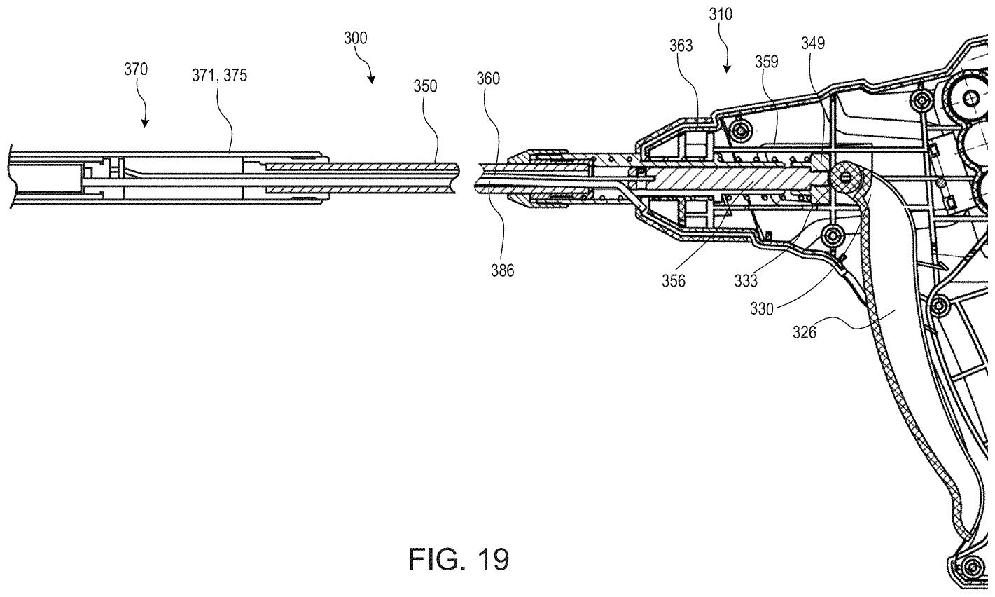

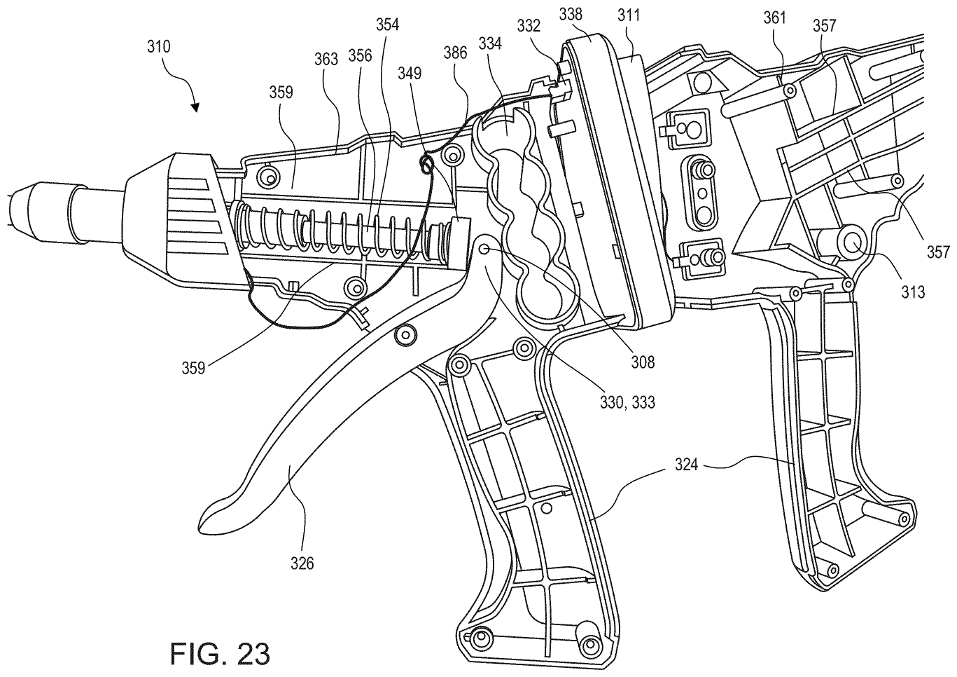

More specifically, as best shown in FIGS. 17 and 21, an annular gap 393 within which a distal portion of the elongate arms 392 are maintained is defined by the second portion 375 of the outer housing 371. In some embodiments the inner surface of the outer housing 371 (e.g., the inner surface of the first housing portion 375 and second housing portion 377) can define one or more guide channels (not shown) as described above for device 100. Such guide channels can have an elongate orientation (i.e., can extend along a longitudinal axis of the distal assembly 370) to guide each of the elongate arms 392 and the inner housing 382 during actuation and movement between a first configuration in which the distal portion of the elongate arms are disposed within the annular gap 393 (as shown, for example, in FIGS. 14 and 17) and a second configuration in which the distal portion of the elongate arms 392 are extended outside of the outer housing 371 (as shown, for example in FIGS. 18 and 21). In other embodiments, the outer housing 371 does not define any guide channels, which arrangement allows the inner housing 382 and/or the elongate arms 392 to rotate relative to the outer housing 371 about the longitudinal axis of the distal assembly 370. In other embodiments, the outer housing 371 can include multiple guide channels (e.g., one for each of the elongate arms 392).

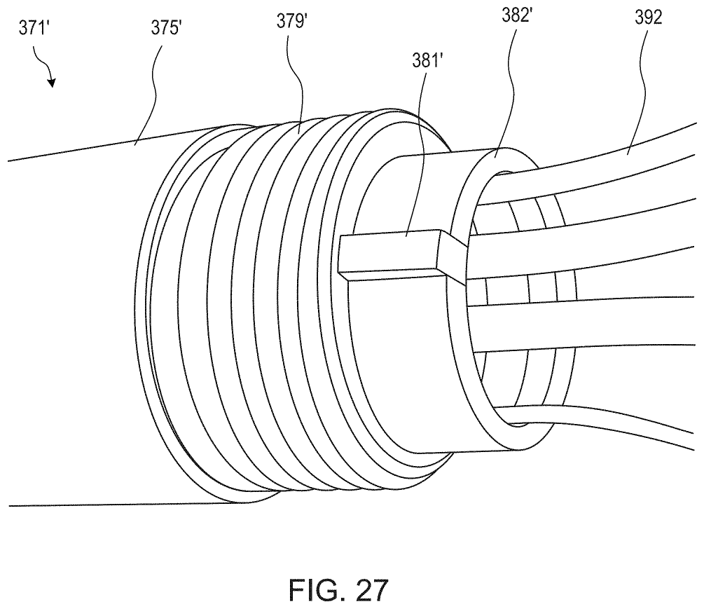

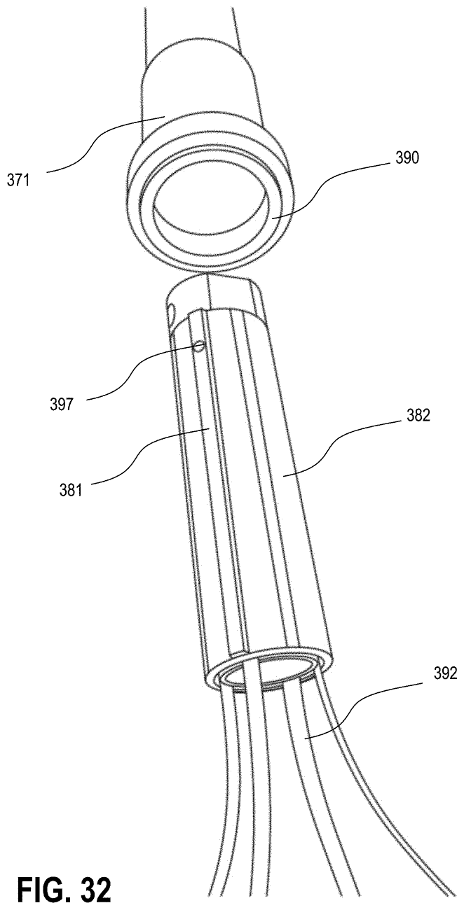

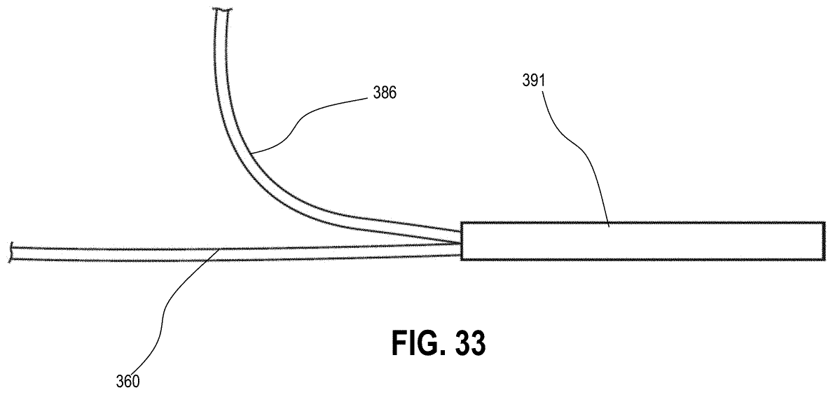

More specifically, in some embodiments, the outer housing 371 can include one or more guide channels defined on an inner surface of the outer housing 371 (e.g., inner surface of the first housing portion 375 or the second housing portion 377, or both the inner surface of the first housing portion 375 and the inner surface of the second housing portion 377) that can slidably receive corresponding elongate protrusion(s) 381 disposed on an outer surface or wall of the inner housing 382 as shown, for example, in FIGS. 31 and 32. Such a guide channel(s) and protrusion(s) 381 can assist in guiding the movement proximally and distally of the inner housing 382 relative to the outer housing 371 during actuation and prevent rotation of the inner housing 382 relative to the outer housing 371. In addition, for manufacturing purposes, the inner housing 382 can include indicia, such as indicia 397 shown in in FIGS. 31 and 32, that can be used to properly align the inner housing 382 with the outer housing 371 during assembly. In this manner, the angular orientation of the electronic device 384 (which is within the inner housing 382) can be maintained as desired when the inner housing 382 is assembled into the outer housing 371. Another such embodiment of an outer housing and an inner housing that have a guide channel and protrusion, respectively, are described below with respect to FIGS. 26-29. In alternative embodiments, the inner housing 382 can include one or more guide channels defined on an outer surface that can slidably receive corresponding elongate protrusions disposed on an inner surface of the outer housing.

The distal end portion of the outer housing 371 includes a magnetic attachment member (also referred to as a magnet) 390 disposed within the second housing portion 377 that can assist with attracting or coupling to a target object. In some embodiments, the magnet 390 can be threadedly coupled to the distal end portion of the outer housing 371, and in other embodiments, a magnet can be coupled to the outer housing 371 by any suitable means, such as by a press fit, an adhesive, or the like. In yet other embodiments, the outer housing 371 need not include the magnetic attachment member 390.

As shown, for example, in FIGS. 17 and 21, the electronic device 384 is disposed within an interior lumen of the inner housing 382. A proximal end portion of the inner housing 382 includes or is coupled to a movable member 372 (which functions as a junction member between the flexible member 360, the electronic device 384, and the elongate arms 392). As shown, for example, in FIGS. 17 and 21, a distal end portion 378 of the movable member 372 is aligned with and coupled to a proximal end portion of the inner housing 382 within the interior of the outer housing 371. As such, the movable member 372 and the inner housing 382 operate together to form a translatable movable carrier (e.g., to carry the electronic device 384 and/or the elongate arms 392) within the outer housing 371. Although the inner housing 382 and the movable member 372 (or junction member) are shown as being separate components that are joined together, in other embodiments, the inner housing 382 and the movable member 372 can be monolithically constructed. A proximal end portion 374 of the movable member 372 is coupled to the distal end portion 368 of the flexible member 360, which extends from the distal end 358 of the flexible shaft 350 and into the interior of the outer housing 371, to couple to the proximal end portion 374 of the movable member 372. The flexible member 360 can be coupled to the movable member 372 with, for example a fastener 351 (see, e.g., FIGS. 17 and 21), which can be a screw or other suitable fastener. As such, the flexible member 360 extends between the movable member 372 and the actuator 326 through the flexible shaft 350, such that movement of the actuator 326 causes the inner housing 382 and the movable member 372 to move together within the outer housing 371 between a first retracted position as shown in FIGS. 14-17 and a second extended position as shown in FIGS. 18-21.

As noted above, the movable member 372 joins the inner housing 382 to the flexible member 360, the electronic device 384, and the elongate arms 392. Specifically, the distal end portion 378 of the movable member 372 includes an elongate arm connection 380 to which the elongate arms 392 are coupled to the movable member 372. In some embodiments, the elongate arm connection 380 shows a portion of each elongate arm 392 being embedded within the movable member 372, and in other embodiments, the elongate arms 392 can be coupled to the movable member 372 and/or the inner housing 382 by any suitable mechanism (e.g., by a weld joint, an adhesive joint, or the like). In some embodiments, the elongate arms 392 can be monolithically constructed with the movable member 372. The proximal end portion 374 of the movable member 372 can also include sealing rings (not shown) disposed around the movable member 372 within the interior volume of the distal housing 371 to retain the movable member 372 in a sliding arrangement within the outer housing 371. The sealing rings can be formed from polymeric materials that provide a low-friction connection within the distal housing to enable sliding movement therein and that also prevent dust, dirt or other foreign materials from entering the interior of the outer housing 371.

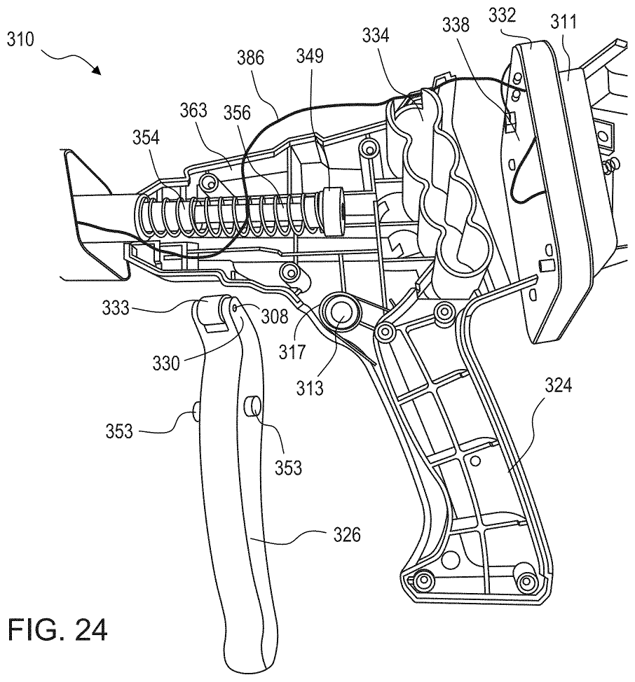

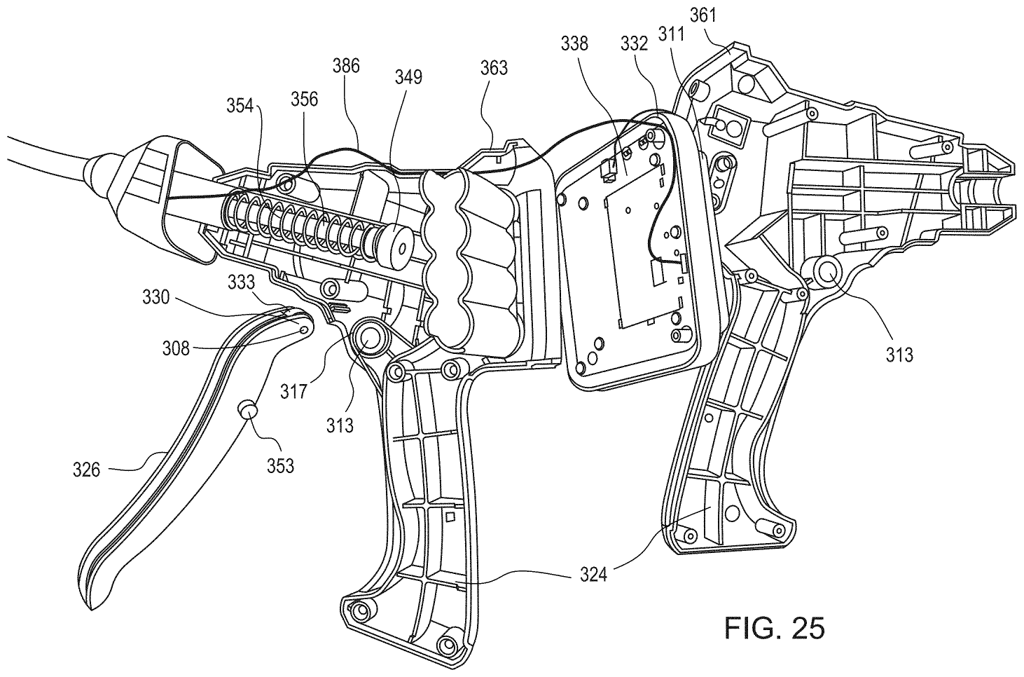

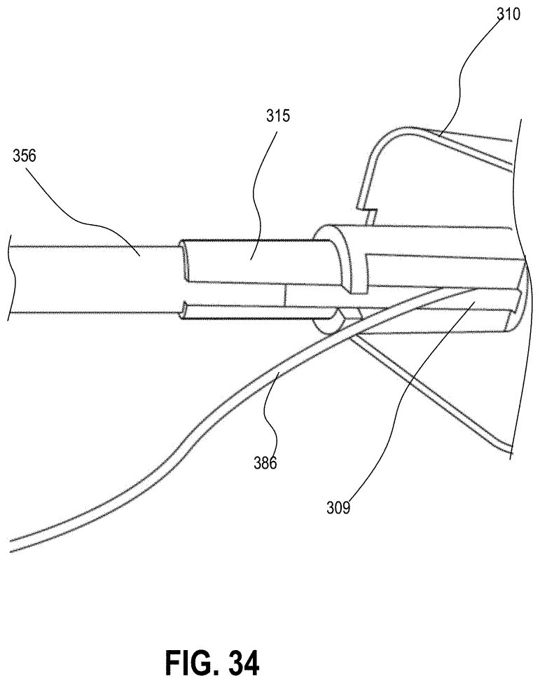

As shown, for example, in FIGS. 14, 17, 18 and 21, the electronic device 384 is attached to the distal end portion 378 of the movable member 372 (and to the inner housing 382) and can be oriented to produce light, capture images, and/or sense conditions when the device is actuated. The electronic device 384 includes a coupling member 388 and an electrical wire 386. The coupling member 388 can include any suitable mechanism for securing the electronic device 384 within a bore of the inner housing 382. For example, in some embodiments, the coupling member 388 can include shock-absorbing properties, an interference fit portion, or any other suitable features to retain the electronic device 384 at the desired position within the inner housing 382. The electrical wire 386 is coupled to the movable member 372 can be part of an electrical assembly that can include one or more wires that extend from the electronic device 384 to an electronics module 332, as shown in FIGS. 23-25 (FIGS. 14, 15, 16, 18, 19 and 20 illustrate a distal end of the electrical wire 386 ending within the housing 312 for illustrative purposes). The electrical wire 386 electrically connects the electronic device 384 to an electronic controller 338 within the electronics module 332 that is coupled to or incorporated within the display device 311. In this embodiment, the electrical wire 386 extends within the flexible shaft 350 alongside the flexible member 360. A portion of the electrical wire 386 and a portion of the flexible member 360 can be coupled together within a single outer wrap 391 (see FIGS. 22 and 33). In other words, the outer wrap 391 can hold or bind together the electrical wire 386 and the flexible member 360 such that the electrical wire 386 and the flexible member 360 can move distally and proximally within the flexible shaft 350 without causing wear or abrasion to the electrical wire 386 and the flexible member 360 that could occur from the two components rubbing along or against each other during movement. For example, a central portion of the flexible member 360 and a central portion of the electrical wire 386 that is disposed within the flexible shaft 350 can be coupled together within the outer wrap 391.

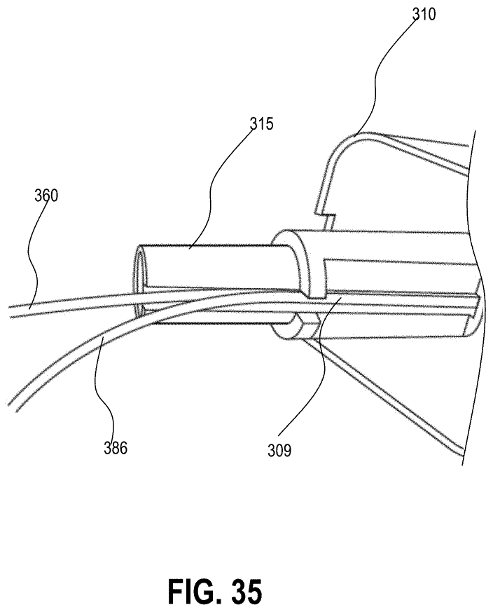

As shown, for example, in FIGS. 34 and 35, a portion of the mounting member 356 is movably coupled within an interior bore of a guide housing 315 of the proximal control assembly 310, as shown in FIG. 34. FIGS. 34 and 35 each illustrate a portion of the proximal control assembly 310, with FIG. 35 showing the mounting member 356 removed from within the guide housing 315 for illustration purposes. The proximal portion of the flexible member 360 and the electrical wire 386 are also routed through the interior bore of the guide housing 315. The guide housing 315 also defines a side opening 309 in communication with the interior bore. The interior bore provides a tight sliding fit for the mounting member 356 and/or the flexible member 360 so as to limit or prevent twisting and/or kinking while moving proximally and distally, and the side opening 309 allows a portion of the electrical wire 386 to extend out of the guide housing 315 and be coupled to the electronics module 332. The electrical wire 386 can allow power to be conveyed from a battery (not shown) disposed within a battery compartment 334 within the proximal housing 312 to the electronic device 384. The electrical wire 386 can also allow control signals and/or data signals to be transferred between the electronic device 384 and the electronic controller 338.

The electronic device 384 can be any suitable device that can produce light and/or sense conditions adjacent the distal end of the device 300. For example, in some embodiments, the electronic device 384 can be a camera, a light emitting device, or an ultrasonic device. In other embodiments, the electronic device 384 can be any sensing device, such as an infrared sensor, a temperature sensor, a radiation sensor, a gas sensor, or an optical sensor. In some embodiments, the electronic device 384 can include (or be coupled to) a wireless network interface configured to transmit a short-range wireless signal associated with an image or a signal received and/or produced by the electronic device 384.

As best shown in FIG. 21, each of the elongate arms 392 includes a proximal end 394 and a distal end 396. Each of the distal ends 396 includes a distal tip 398. As described above, the proximal end 394 of each of the plurality of elongate arms 392 is coupled to an outer portion of the movable member 372 at the coupling portion 380 of the movable member 372. Each of the elongate arms 392 extends from the respective proximal end 394 to the respective distal end 396 of the elongate arm. As can be seen in, for example, FIG. 17, the distal end 396 of each elongate arm 392 is configured to be at a first position within the outer housing 371 when the inner housing is in a first, non-deployed position. When the elongate arms 392 are in the first position, the distal tip 398 of each elongate arm 392 is disposed or retracted within the interior of the outer housing 371. When the elongate arms 392 are in the first position, they are also in a first (deformed) configuration. Specifically, each of the elongate arms 392 is urged inwardly by an inner wall of the outer housing 371.

As shown, for example, in FIG. 21, when the device 300 is actuated, the distal tip 398 of each elongate arm 392 is at a second position extending outside of the outer housing 371. More specifically, because the elongate arms 392 are coupled to the inner housing 382 (via the movable member 372), upon actuation of the actuator 326, the movable member 372 and inner housing 382 are moved within the outer housing 371 via the flexible member 360, and the elongate arms 392 are moved relative to the outer housing 371 to a second deployed position. When the elongate arms 392 are in the second deployed position, the distal tip 398 of each elongate arm 392 extends from the distal-most surface of the outer housing 371.

The elongate arms 392 can be made from a flexible material (e.g., spring steel) that is arranged to rotate or flex outward away from each other and away from their longitudinal direction as they are translated outward of the interior of the outer housing 371 during actuation of the device 300. The distal ends 396 are arranged to form a set of inward directed hook-like shapes at each tip 398. As such, when the actuator 326 is actuated, the tip 398 of each elongate arm 392 is directed inward toward a central region defined by the elongate arms 392 and a central point between the tips 398. In this manner, when the device 300 is returned to its undeployed state, the tips 398 can grasp an object within the central region.

In addition to moving the elongate arms 392 between their first position and their second position, actuation of the device 300 also moves the electronic device 384 between a first position at a first location within the outer housing 371 as shown, for example, in FIG. 17, to a second position at a second location within the outer housing 371, as shown, for example, in FIG. 21. Specifically, because the electronic device 384 is fixedly coupled within the inner housing 382, movement of the inner housing 382 and the movable member 372, which causes movement of the elongate arms 392, also causes the electronic device 384 to be moved from its first position to its second position.

The relative position of the electronic device 384 and the tips 398 when the elongate arms 392 are deployed can cooperatively function to provide advantageous data collection. For example, as described herein, in embodiments in which the electronic device 384 is a camera, the camera can receive an image that includes an end portion 396 of the elongate arms 392. In addition, the electronics module 332 can be configured to identify the tips 398 in the image. The electronics module 332 can further be configured to identify the central point between the tips 398 and a line segment between each tip 398 and the central point, and to show the line segments and/or central point as virtual features in the display screen 319 (see FIG. 13B) of the display device 311 as discussed further below.

As described above, in this embodiment, the display device 311 is incorporated within the proximal control assembly 310 and is permanently or fixedly coupled thereto. The display device 311 includes a viewer application configured to connect with the electronic controller 338 of endoscopic grabber device 300 and display information received from the electronic device 384. The viewer application can be configured simply to display a view provided from the electronic controller 338 and/or the viewer application can be configured to modify the view as discussed further below, such as to add virtual line segments to provide a cross-hairs type view, show a virtual central point and/or to estimate distances to the tips 398 or the target object.

In some embodiments, the display device 311 displays an image that is captured by the electronic device 384. The image can show the tips 398 of the elongate arms 392 along with the target object and corresponding environmental features within its view while the endoscopic grabber device 300 is activated and being used. The electronic controller 338 can be configured to identify the tips 398 in the captured image. Alternatively, the viewer application can be configured to identify the tips 398 in the captured image, and the tips 398 could optionally be highlighted or marked to aid the identification. The viewer application or the electronic controller 338 can further be configured to identify the central point between the tips and a line segment between each tip and the central point, and to show the line segments and/or central point as virtual features on the display device 311. These virtual features could provide a cross-hairs type view to aid the user with aligning the endoscopic grabber device 300 effectively for grabbing the target object.

In addition, the electronic controller 338 and/or the viewer application can be configured to estimate distances to the target object and/or the distance that the elongate arms 392 extend from distal end of the endoscopic grabber device 300. The size of the tips 398 can be known to the electronic controller 338 and/or the viewer application, which can be used to determine the distance that the tips are extended. Further, the electronic controller 138 and/or viewer application can compare the size of the target object being viewed with the size of the tips, as well as monitor the changing size of the object when approaching the target object, from which distances can be estimated. In addition, the distance that the elongate arms are extended can be monitored based on movement of the movable member, for example, which can provide additional information for estimating distances and/or the size of the target object.

As best seen in FIGS. 14-16 and 18-20, the control assembly 310 includes the proximal housing 312 formed with a first housing part 361 and a second housing part 363, a rear handle or grip portion 324, a shaft connection 355, a manipulator portion 322 that receives the proximal portion 352 of the flexible member 360, the actuator or trigger 326, and the electronics module 332. The proximal housing 312 defines an internal volume 314 when the first and second housing parts 361 and 363 are coupled together in which the electronics module 332 is secured. The first housing 361 includes walls 357 and second housing part 363 includes walls 359. When the housing parts 361 and 363 are coupled together a guide passage 367 is defined by the walls 357 and 359 in which the mounting member 356 travels distally and proximally. More specifically, as described above, the mounting member 356 includes the guide portion 349 at a proximal end. The guide portion has an outer diameter or width that is smaller than a diameter or width of the guide passage 367 such that the guide portion 349 can travel distally and proximally within the guide passage 367. The walls 357 and 359 help guide the guide portion 349 and mounting member 356 as it travels distally and proximally upon actuation, and help maintain a stable transition between the retracted and extended positions of the flexible member 360.

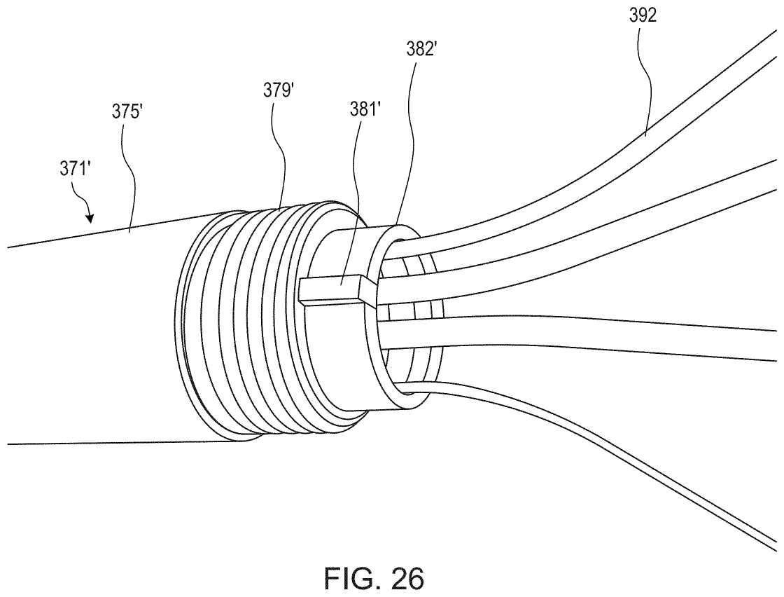

The rear handle or grip portion 324 is configured as a handle that can be easily held by a user and allow the user to manipulate the endoscopic grabber device 300 during use. The actuator or trigger 326 is disposed on an upper, front region of the grip portion 324 and located for easy access by a user's index finger. The actuator or trigger 326 is coupled to the housing 312 via pivot pins 353 (see FIGS. 24 and 25) at a pivot portion 328 of the actuator 326 as described above. The pivot pins 353 are rotatably mounted within the pivot shafts 313 defined by the first and second housing parts 361 and 363 (see FIGS. 24 and 25) in the internal volume 314 of the proximal housing 312 when coupled together. A torsion spring 317 (see, e.g., FIGS. 18, 24 and 25) is coupled between the actuator 326 and the housing 312 about the pivot shaft 313. A lever end 330 of the actuator 326 is located on an internal end of the pivot portion 328 adjacent to the mounting member 356 at the proximal end portion 362 of the flexible member 360. The lever end 330 is arranged in a cantilever arrangement with the exposed trigger end of the actuator 326 on the pivot portion 328. Actuation of the trigger end of the actuator 326 rotates the pivot portion 328 (i.e., the pivot pins 353 of the actuator 326 rotate within the pivot shaft 313 of the housing 312) and causes the lever end 330 to rotate in a direction toward the mounting member 356 and the distal assembly 370. Thus, the lever end 330 engages the guide portion 349 of the mounting member 356 and pushes the flexible member 360 causing it to translate distally within the flexible shaft 350 in the direction of the distal assembly 370. More specifically, the lever end 330 includes a curved engagement portion 333 that contacts the guide portion 349 upon actuation (see e.g., FIGS. 24 and 25). In this embodiment, the engagement portion 333 is a roller that is pivotally or rotatably coupled to the actuator 326 via a pin(s) 308 (see, e.g., FIG. 23) such that the engagement portion 333 can rotate as it contacts the guide portion 349. This rotation and the curved shape of the engagement portion assist in maintaining good contact with the guide portion 349 of the mounting end 356 during actuation and assist in a smooth engagement between the engagement portion 333 of the lever end 330 and the guide portion 349 of the mounting end 356.

The shaft connection 355 is disposed on a forward portion of the proximal housing 312 to securely connect the proximal end portion 352 of the flexible shaft 350 to the proximal housing 312. The manipulator portion 322 is disposed within the shaft connection 355 to receive the proximal end portion 352 of the flexible shaft 350 and to retain components of the proximal end portion of the flexible shaft 350, which are discussed in more detail below along with the flexible shaft 350.

The electronics module 332 include can include a control switch (not shown), the electronic controller 338 (which can include a processor), and one or more lights (not shown). As described above, a power source can include batteries within the battery compartment 334 defined within the proximal housing 312. In other configurations, the power source can include components for coupling to an alternating current power supply (not shown) in addition to components for a battery power source or as an alternative to a battery power source. Such configurations can include a power cord and transformer, as well as a charger for charging a battery. The control switch can include a simple on/off switch, as well as optional settings for activating the lights and/or the electronic device 384. The electronics controller 338 can include a processor, a memory, and a wireless network interface.

The processor can be configured to run and/or execute application modules, processes and/or functions associated with the device 300. For example, the processor can be configured to run and/or execute an image capture module that facilitates capturing and processing of an image produced by the electronic device 384. The processor can be, for example, a Field Programmable Gate Array (FPGA), an Application Specific Integrated Circuit (ASIC), a Digital Signal Processor (DSP), and/or the like. The processor can be configured to retrieve data from and/or write data to a memory device (not shown). As described herein, in some embodiments, the processor can cooperatively function with the network interface device and/or a radio to provide signals to communicatively couple the electronics module 332 to a remote computing device via a wireless communication and/or any other computing entity via a network. In some embodiments, the processor is a Bluetooth® low energy (BLE) processor, such as The Texas Instruments® CC2540 series of processors, the Broadcom® BCM43341 processor, and/or any other processor suitable or configured specifically to execute the Bluetooth® v4.0 low energy stack.

The memory (not shown) can be, for example, random access memory (RAM), memory buffers, hard drives, databases, erasable programmable read only memory (EPROMs), electrically erasable programmable read only memory (EEPROMs), read only memory (ROM), flash memory, hard disks, floppy disks, cloud storage, and/or so forth. In some embodiments, the memory stores instructions to cause the processor to execute modules, processes and/or functions associated the device 300. For example, the memory can store instructions to cause the processor to execute the image capture module.

The electronic controller 338 can include a wireless interface or radio, which can be any suitable communication device and can be a part of the overall processor architecture of the electronic controller 338, (e.g., a part of a Bluetooth® processor). In other embodiments, the radio or wireless interface can be distinct from a processor of the electronic controller 338. In some embodiments, a short-range radio link can be established between the electronic module 332 and a mobile electronic device. For example, the electronic module 332 and/or the electronic controller 338 and a remote computing device can be paired via the Bluetooth® wireless protocol. Similarly stated, the electronic module 332 and/or the electronic controller 338 and the remote computing device can be paired via a wireless protocol that facilitates the transmission of signals within a range of approximately 700 meters or less (i.e., a Class 3 radio) and/or having a frequency within the range of 2400 MHz and 2480 MHz. In such an embodiment, the electronics module 332 and/or the electronic controller 338 can be operable to send and/or receive data from the display device 311 related to an image acquire by the device, such as from the electronic device 384 to a remote computing device (e.g., a phone, tablet, computer, etc.).

In operation, actuation of the actuator 326 moves the mounting member 356 within the proximal housing 312 as discussed above to push the flexible member 360 distally within the flexible shaft 350 toward the distal assembly 370. Movement of the flexible member 360 correspondingly moves the movable member 372 forward within the distal housing 371, which advances the elongate arms 392 and the electronic device 384 distally within the distal assembly 370 as previously described. As the elongate arms 392 advance and extend outside of the outer housing 371, the elongate arms 392 flex or rotate outward to an expanded biased configuration which increases the size of a central region disposed between the tips 398 of the elongate arms 392. In some embodiments, the advancement of the electronic device 384 also allows the central region produced between the tips 398 to be sensed by the electronic device 384 (e.g., viewed by the camera, in some embodiments). The endoscopic grabber device 300 can be advanced toward a target object (not shown) based on the camera view to place the target object within the central region. The user can release the actuator 326 which allows the spring 354 to bias the flexible member 360 proximally, moving the elongate arms 392 proximally partially within the distal housing 371, thereby collapsing the tips 398 around the target object to grab the object. If the target object is magnetic, the optional magnet 390 can be used to grab the object via a magnetic connection alone or along with use of the elongate arms 392.

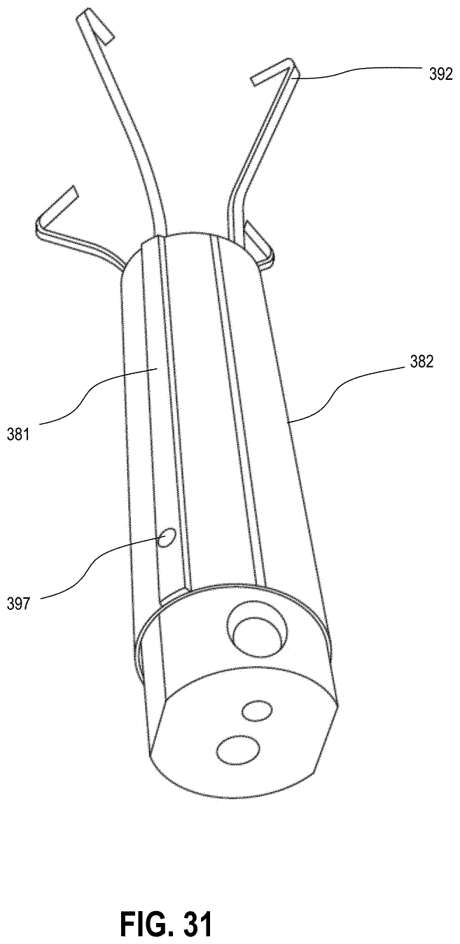

FIGS. 26-30 illustrate an alternative embodiment of an outer housing 371′ and an inner housing 382′ that can be used in (incorporated within) the endoscopic grabber 300 or any of the endoscopic grabbers shown herein. The outer housing 371′ defines an interior volume within which the inner housing 382′ can be movably disposed. The outer housing 371′ can include a first housing portion 375′ threadably coupled to a second housing portion 377′ (see cross-sectional view of FIG. 30) at a threaded coupling joint 379′. In this embodiment, the outer housing 371′ includes a guide channel 369′ (see cross-sectional view of FIG. 30) defined on an inner surface of the outer housing 371′ (e.g., an inner surface of the first housing portion 375 and an inner surface of the second housing portion) that extends longitudinally along the inner surface of the outer housing 371′. The guide channel 369′ can slidably receive an elongate protrusion 381′ disposed on an outer surface of the inner housing 382′.

During actuation of the device 300, the guide channel 369′ on the outer housing 371′ and protrusion 381′ on the inner housing 382 can assist in guiding the movement proximally and distally of the inner housing 382′ relative to the outer housing 371′ and prevent rotation of the inner housing 382′ relative to the outer housing 371′. As such, during actuation, the movement of the elongate arms 392 and camera 384, which are coupled to the inner housing 382′, can translate proximally and distally without rotating, which can prevent or limit possible binding of the device 300. For example, the camera 384 and electrical wire 386 coupled thereto can be maintained aligned longitudinally with the flexible member 360.

In some embodiments, only the first housing portion 375′ of the outer housing 371′ includes a guide channel to receive a protrusion on the inner housing 382′. In such an embodiment, the second housing portion can include a larger diameter such that the inner housing 382′ along with the protrusion 381′ can slidably move proximally and distally within the interior volume of the second housing portion. In some embodiments, the inner housing 382′ can include a guide channel defined on an outer surface that can slidably receive an elongate protrusion disposed on an inner surface of the outer housing 371′ (e.g., an inner surface of the first housing portion 375′ and second housing portion).

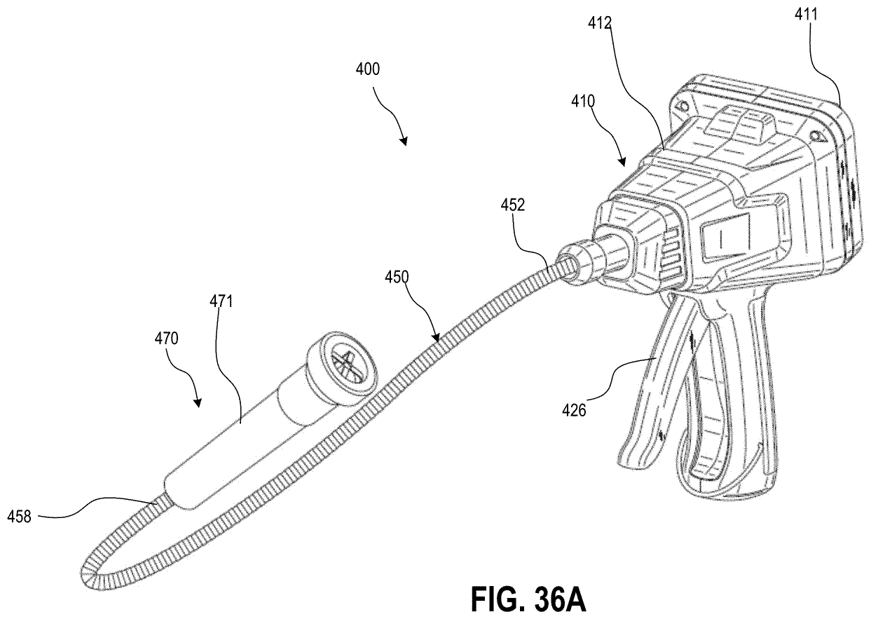

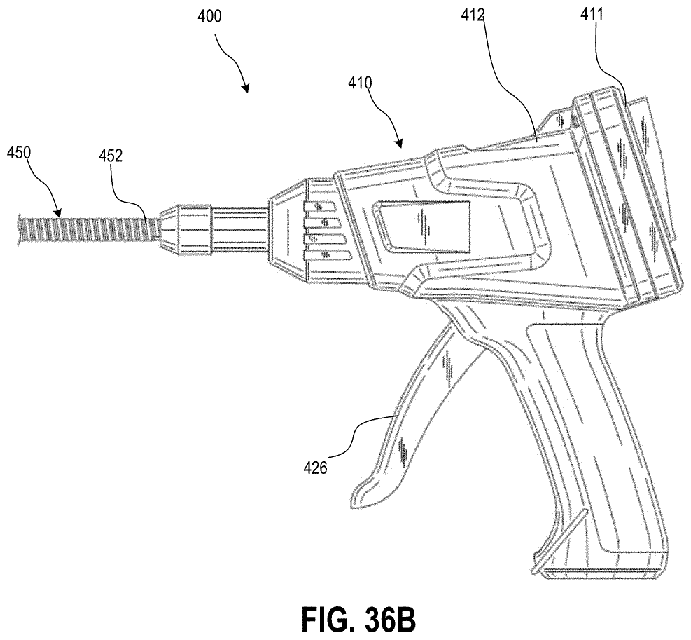

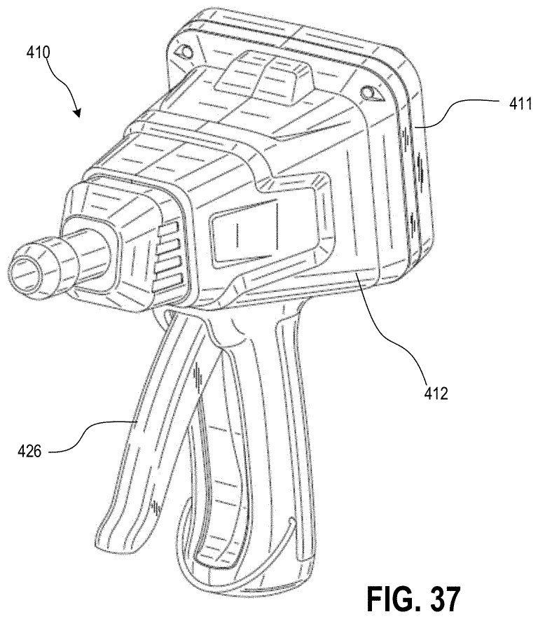

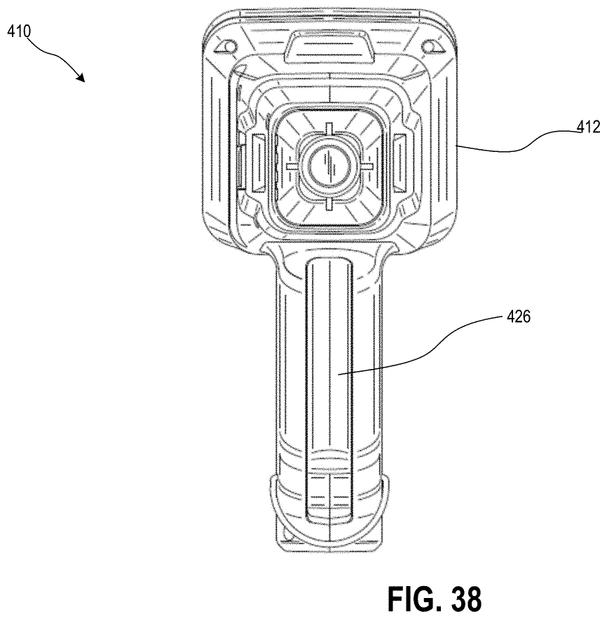

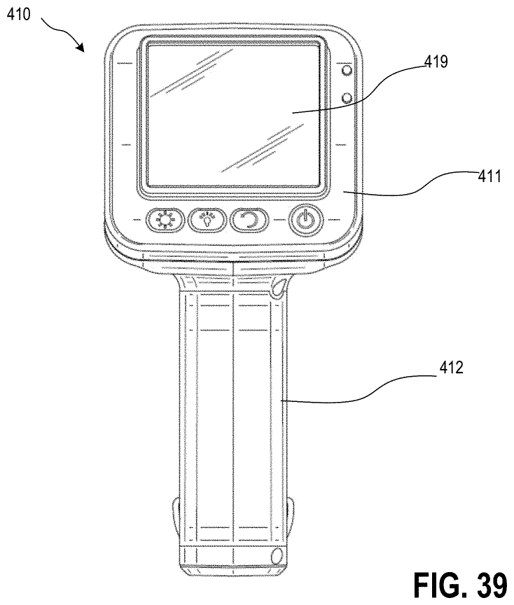

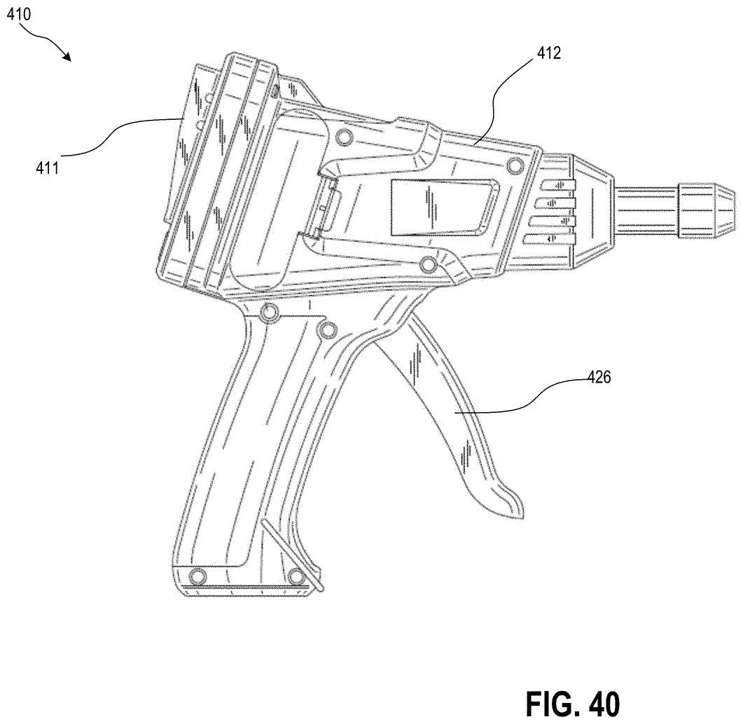

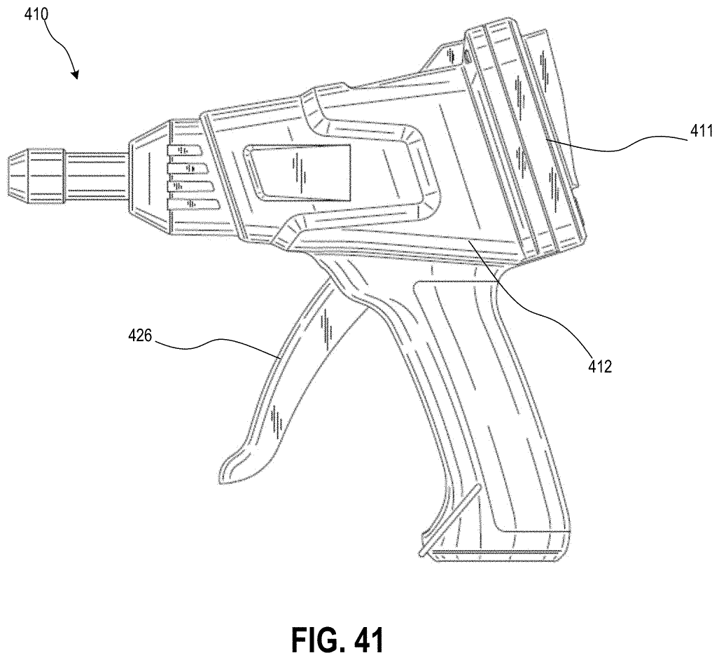

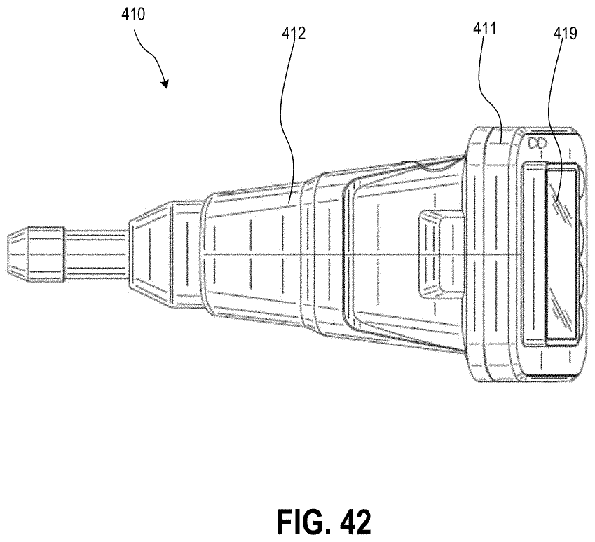

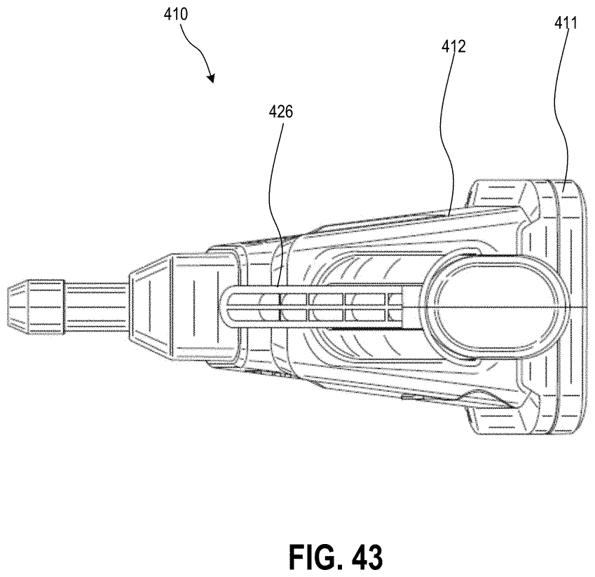

FIGS. 36A-43 illustrate another embodiment of an endoscopic grabber. An endoscopic grabber 400 can include the same or similar features and functions as described above for endoscopic grabber 100 and endoscopic grabber 300, and therefore some features and functions are not described in detail with reference to this embodiment.

The endoscopic grabber device 400 includes a control assembly 410 having a proximal housing 412 and the control components therein, including an electronics module (not sown), a distal assembly 470, and a flexible shaft 450 extending between the control assembly 410 and the distal assembly 470. The flexible shaft 450 has a distal end portion 458 connected to the distal assembly 470, and a proximal end portion 452 connected to the proximal housing 412. A flexible member (not shown) is located within the flexible shaft 450. In this embodiment, the control assembly 410 also includes a viewing device 411 coupled to the proximal housing 412.

The control assembly 410 also includes an actuator 426 coupled to the proximal housing 412 via a pivot pin (not shown) located at a pivot portion (not shown) and that is received within a pin shaft (not shown) of the housing 412 such that the actuator 426 can pivot relative to the housing 412. A torsion spring (not shown) is coupled between the actuator 426 and the housing 412. The actuator 426 is configured to actuate movement of the flexible member as described above for endoscopic grabbers 300 and 300′. The flexible member is movably disposed within the flexible shaft 450. A distal end portion of the flexible member is coupled to the distal assembly 470, and a proximal end portion of the flexible member is coupled to a mounting member within the proximal housing 412. The actuator 426 is configured to move the flexible member via the mounting member to actuate movement of the distal assembly 470 as described above for previous embodiments.

In some embodiments, the flexible member can be formed as a flexible wire. The flexible shaft 450 can be formed from a flexible metal shaft, a flexible elastomeric shaft, or the like, and defines an internal channel for the flexible member to translate therein while permitting the shaft to bend and flex as needed to reach a target object during use. As described above, the mounting member is disposed at the proximal end portion of the flexible member and movably disposed within the flexible shaft 450. The mounting member includes a guide portion at a proximal end that engages the actuator and travels within a guide passage of the proximal housing 412, as described above for previous embodiments. A spring (not shown) is disposed about and coupled to the mounting member within the proximal housing 412. The spring biases the flexible member in a direction of the proximal housing 412 as shown in FIGS. 36-43. Upon actuation of the actuator 426, the mounting member moves against the bias of the spring, and the mounting member and flexible member are urged in a direction toward the distal assembly 470. Thus, actuation of actuator 426 advances the flexible member into an extended position, and releasing the actuator 426 biases the flexible member into a retracted position, as shown and described above for example, for endoscopic grabber 300.

The distal assembly 470 includes an outer housing 471, an inner housing (not shown) movably disposed within the outer housing 470, multiple elongate arms (not shown) coupled to the inner housing, and an electronic device (not shown) disposed within the inner housing 471. The distal assembly 470 can be constructed the same as or similar to and function the same as or similar to the distal assembly 370 described above, and therefore, certain details of the distal assembly 470 are not provided.

The electronic device can be any suitable device that can produce light and/or sense conditions adjacent the distal end of the device 400. For example, in some embodiments, the electronic device can be a camera, a light emitting device, or an ultrasonic device. In other embodiments, the electronic device can be any sensing device, such as an infrared sensor, a temperature sensor, a radiation sensor, a gas sensor, or an optical sensor. In some embodiments, the electronic device can include (or be coupled to) a wireless network interface configured to transmit a short-range wireless signal associated with an image or a signal received and/or produced by the electronic device.

A distal end of each elongate arm is configured to be at a first position within the outer housing 471 when the flexible member is biased in the direction of the proximal housing 412. This is a first, non-deployed position. When the elongate arms are in the first non-deployed position, a distal tip of each elongate arm is disposed or retracted within the interior of the outer housing 471. When the elongate arms are in the first position, they are also in a first (deformed) configuration. Specifically, each of the elongate arms is urged inwardly by an inner wall of the outer housing.

When the device 400 is actuated, the distal tip of each elongate arm is at a second position extending outside of the outer housing 471. More specifically, because the elongate arms are coupled to the inner housing, upon actuation of the actuator 426, the movable inner housing is moved within the outer housing 471 via the flexible member, and the elongate arms are moved relative to the outer housing 471 to a second deployed position. When the elongate arms are in the second deployed position, the distal tip of each elongate arm extends from the distal-most surface of the outer housing 471.

The elongate arms can be made from a flexible material (e.g., spring steel) that is arranged to rotate or flex outward away from each other and away from their longitudinal direction as they are translated outward of the interior of the outer housing 471 during actuation of the device 400. The distal ends are arranged to form a set of inward directed hook-like shapes at each tip. As such, when the actuator 426 is actuated, the tip of each elongate arm is directed inward toward a central region defined by the elongate arms and a central point between the tips. In this manner, when the device 400 is returned to its undeployed state, the tips can grasp an object within the central region.

In addition to moving the elongate arms between their first position and their second position, actuation of the device 400 also moves the electronic device between a first position at a first location within the outer housing, to a second position at a second location within the outer housing. Specifically, because the electronic device is fixedly coupled within the inner housing, movement of the inner housing, which causes movement of the elongate arms, also causes the electronic device to be moved from its first position to its second position.

The relative position of the electronic device and the tips when the elongate arms are deployed can cooperatively function to provide advantageous data collection. For example, as described herein, in embodiments in which the electronic device is a camera, the camera can receive an image that includes an end portion of the elongate arms. In addition, the electronics module can be configured to identify the tips in the image. The electronics module can further be configured to identify the central point between the tips and a line segment between each tip and the central point, and to show the line segments and/or central point as virtual features in a display screen 419 of the display device 411.

As described above, in this embodiment, the viewing device 411 is incorporated within the proximal control assembly 410 and permanently or fixedly coupled thereto. The viewing device 411 includes a viewer application configured to connect with an electronic controller within the electronics module and display information received from the electronic device. The viewer application can be configured simply to display a view provided from the electronic controller and/or the viewer application can be configured to modify the view as discussed further below, such as to add virtual line segments to provide a cross-hairs type view, show a virtual central point and/or to estimate distances to the tips or the target object.

The viewing device 411 and the electronic controller can be configured the same as or similar to and function the same as or similar to the electronic controller 338 described above, and is, therefore not described in further detail here. Similarly, the viewer application can be configured the same as or similar to, and function the same as or similar to, the viewer application described above for device 300. The control assembly 410 can also be configured the same as or similar to and function the same as or similar to, the control assembly 310 described above, and is therefore not described in detail here.

In operation, actuation of the actuator 426 moves the mounting member within the proximal housing 412 as discussed above for device 300 to push the flexible member distally within the flexible shaft 450 toward the distal assembly 470. Movement of the flexible member correspondingly moves the inner housing within the outer housing 471, which advances the elongate arms and the electronic device distally within the distal assembly 470 as previously described for device 300. As the elongate arms advance and extend outside of the outer housing 471, the elongate arms flex or rotate outward to an expanded biased configuration which increases the size of a central region disposed between the tips of the elongate arms. In some embodiments, the advancement of the electronic device also allows the central region produced between the tips to be sensed by the electronic device (e.g., viewed by the camera, in some embodiments). The endoscopic grabber device 400 can be advanced toward a target object (not shown) based on the camera view to place the target object within the central region. The user can release the actuator 426 which allows the spring to bias the flexible member proximally, moving the elongate arms proximally partially within the outer housing 471, thereby collapsing the tips around the target object to grab the object. If the target object is magnetic, an optional magnet can be used to grab the object via a magnetic connection alone or along with use of the elongate arms.

While various embodiments of the invention have been described above, it should be understood that they have been presented by way of example only, and not limitation. Where methods described above indicate certain events occurring in certain order, the ordering of certain events may be modified. Additionally, certain of the events may be performed concurrently in a parallel process when possible, as well as performed sequentially as described above.

For example, although the electronic device (e.g., 184, 284, 384) is shown as moving along with the movable member (e.g., 172, 272, 372), in other embodiments, the electronic device (e.g., 184, 284, 384) can remain stationary within the distal assembly (e.g., 170, 270, 370, 470) when the movable member (e.g., 172, 372) moves. For example, in some embodiments, an endoscopic grabber can include an electronic device that is fixedly mounted to an outer housing and does not move when the elongate arms of a grabber extend from the device. In other embodiments, an endoscopic grabber can include an electronic device that remains stationary relative to the elongate arms during a first portion of the extension, and then moves along with the elongate arms during a second portion of the extension.

Although the electronic device (e.g., 184, 284, 384) is shown as being coupled to an electronic controller (e.g., 138, 238, 338) via a wire, and the electronic controller is shown as transmitting a wireless signal to an image display device (e.g., viewing device), in other embodiments, any suitable mechanisms for coupling the electronic device (e.g., 184, 284, 384) to an image display device can be used. For example, in some embodiments, an electronic device mounted within a distal assembly can include a radio and can therefore be coupled directly to the display device without first being coupled to a controller. In other embodiments, a display device can be coupled to the controller via a wired coupling.

Although various embodiments have been described as having particular features and/or combinations of components, other embodiments are possible having a combination of any features and/or components from any of embodiments where appropriate. For example, although some embodiments are described as having a processor, a radio, a sensor, etc. disposed on a particular portion of a device, in other embodiments, any of the electronic circuit systems can be disposed on any suitable portion of an endoscopic device.

What is claimed is:

1. An apparatus comprising:
a proximal control assembly including a proximal housing and an actuator;
a distal assembly including:
an outer housing;
an inner housing movably disposed within the outer housing;
an electronic device disposed within a bore of the inner housing; and
a plurality of elongate arms coupled to the inner housing such that at least a portion of each of the plurality of elongate arms is disposed between an inner surface of the inner housing and the electronic device;
a flexible member having a distal end portion coupled to the inner housing, and a proximal end portion coupled within the control assembly;
an electrical wire having a distal end portion coupled to the electronic device and proximal end portion coupled within the control assembly; and
actuation of the actuator configured to move the flexible member to cause the inner housing to move within the outer housing between a first position and a second position, a distal end portion of each elongate arm from the plurality of elongate arms being in a first configuration within the outer housing when the inner housing is in the first position and in a second configuration outside of the outer housing when the inner housing is in the second position, and simultaneously a distal end of the electronic device being in a first location within the outer housing when the inner housing is in the first position, the distal end of the electronic device being in a second location within the outer housing when the inner housing is in the second position, the second location being distal of the first location.

2. The apparatus of clam 1, wherein the electronic device is a camera, the camera configured to capture an image including a portion of an elongate arm from the plurality of elongate arms.

3. The apparatus of claim 2, further comprising:
a display screen fixedly coupled to the proximal housing of the control assembly, the display screen configured to display the image captured by the camera.

4. The apparatus of claim 1, further comprising:
a flexible shaft having a distal end portion and a proximal end portion, the proximal end portion of the flexible shaft coupled to the proximal control assembly, the distal end portion of the flexible shaft coupled to the outer housing, the flexible member movably disposed within the flexible shaft.

5. The apparatus of claim 1, further comprising:
a guide housing coupled within the proximal housing and defining an interior bore and a side opening in communication with the interior bore, a portion of the mounting member and the proximal portion of the flexible member each movably disposed within the interior bore of the guide housing,
the electrical wire having a first portion movably disposed within the interior bore of the guide housing and a second portion extending out through the side opening, the second portion of the electrical wire coupled to an electronics module coupled to the proximal housing.

6. The apparatus of claim 5, further comprising:
a mounting member having a guide portion , the mounting member disposed within the proximal housing, the actuator operatively coupled to the guide portion, the proximal end of the flexible member being coupled to the mounting member such that actuation of the actuator applies a force against the guide portion, causing the mounting member and the flexible member to move distally within the guide housing.

7. The apparatus of claim 6, wherein the actuator includes a roller configured to contact the guide portion of the mounting member causing the mounting member and the flexible member to move distally within the guide housing.

8. The apparatus of claim 1, wherein:
the inner housing includes at least one protrusion on the outer surface of the inner housing; and
an inner surface of the outer housing defines at least one guide channel , each protrusion from the at least one protrusion configured to slidably move in a different guide channel from the at least one guide channels when the actuator is actuated to prevent rotation of the inner housing relative to the outer housing.

9. The apparatus of claim 1, wherein a portion of the flexible member and a portion of the electrical wire are coupled together within an outer wrap.

10. An apparatus comprising:
a proximal control assembly including a proximal housing and an actuator;
a distal assembly including:
an outer housing;
an inner housing movably disposed within the outer housing;
a plurality of elongate arms coupled to the inner housing; and
a camera disposed within a bore of the inner housing and configured to capture an image including a portion of an elongate arm from the plurality of elongate arms;
a display screen fixedly coupled to the proximal housing of the control assembly, the display screen configured to display the image captured by the camera;
a flexible member having a distal end portion coupled to the inner housing, and a proximal end portion coupled within the control assembly;
an electrical wire having a distal end portion coupled to the camera and proximal end portion coupled within the control assembly, a central portion of the electrical wire coupled to a central portion of the flexible member; and
actuation of the actuator configured to move the flexible member to cause the inner housing to move distally within the outer housing such that the plurality of elongate arms and the camera both move distally within the outer housing and a portion of the plurality of elongate arms moves outside of the outer housing.

11. The apparatus of claim 10, further comprising:
a flexible shaft having a distal end portion and a proximal end portion, the proximal end portion of the flexible shaft coupled to the proximal control assembly, the distal end portion of the flexible shaft coupled to the outer housing, the flexible member movably disposed within the flexible shaft.

12. The apparatus of claim 10, further comprising:
a guide housing coupled within the proximal housing and defining an interior bore and a side slot in communication with the interior bore, a portion of the mounting member and the proximal portion of the flexible member each movably disposed within the interior bore of the guide housing,
the electrical wire having a first portion movably disposed within the interior bore of the guide housing and a second portion extending out through the side slot, the second portion of the electrical wire coupled to an electronics module coupled to the proximal housing.

13. The apparatus of claim 12, further comprising:
a mounting member having a guide portion, the mounting member disposed within the proximal housing, a portion of the actuator operatively coupled to the guide portion, the proximal end of the flexible member being coupled to the mounting member such that actuation of the actuator applies a force against the guide portion, causing the mounting member and the flexible member to move distally within the guide housing.

14. The apparatus of claim 13, wherein the actuator includes a roller configured to contact the guide portion of the mounting member causing the mounting member and the flexible member to move distally within the guide housing.

15. The apparatus of claim 10, wherein:
the inner housing includes at least one protrusion on the outer surface of the inner housing; and
an inner surface of the outer housing defines at least one guide channel, each protrusion from the at least one protrusion configured to slidably move within a different guide channel from the at least one guide channels when the actuator is actuated to prevent rotation of the inner housing relative to the outer housing.

16. The apparatus of claim 10, wherein the plurality of elongate arms are coupled to the inner housing such that at least a portion of each of the plurality of elongate arms is disposed between an inner surface of the inner housing and the camera.

17. The apparatus of claim 10, wherein the central portion of the flexible member and the central portion of the electrical wire are coupled together within an outer wrap.
